SEQUENCE LISTING (10 sequences)

SEQ ID NO: 1 - DNA, 49396 bp, Homo sapiens
taagaaacttcttagattgccctgaattctaaagaatctttctcattttgagtttgtgacatttcaatgtgagaaaattaacggaagctttagaggctgttattcaagcttagcattttttagtgcaagcgtcataaattttgtagctacagtgatttaaatttatctattttacaaagtctttggaaagccatttcctttctctggacagtgggagagaatatttggaagccagtgatctgaaaacagacatgaactgttttctggtcttctgtaccctacagaacagtaatcaaagttgcaaaagttagacttatcaaagtcttaacaactgcggcatcactcggaaagcccttcaacggccccaaacatggctttgagtttggacctgttctttgtcttaaaagaattatgtttggcaagccaaaaaagctttgcatgtatctaatgctcagaggcttaaaggcctcagctctatattttcataatgtctgggcttaattttgaaagattttaaaatctggctttaattttaaatgtgatagatcatcaaggatatttttccagggagcaagccaccaaaataaatgcaattccaaataaagttaacagcaacactcaaccaatgttcaccctactcagacagattgttcattgaaatttgattttgacagtattttctaatgtttacgtgacacataatacatttcagaacattagacatcctttatatatgtacttgaggaagaatccagaaggatgctcggagaactggttattttagaaactttacaaaattggcctttcgtttgtgaaacttgaatcttattacacatgatatagagtttggtcacatggactgtgtccattaatcttaagaaacatgattttgactaaagtaaccatatacattttctgagttttggttcttgcttgagagatgcctacaccttgactgcttaggatatgtagcagaacacacaggatttagaaattagagggtggagattgtagtaaacatattttaatgcctgttcagtttgagacacacaaagttttaagttttgaaaagaagaaagttcagaaattatttgaaatatgagtaagccactaatatttcttgatctcagttttccttagtaagtaaaatgaggcgtagggtacaattattttaaagatatgttctaaatgtgagattgtgtgaccttattagagaaaacagtagaattaaaagaaaaaatactaagtcaggaagaagaggatttgggatagtgcagtatcatagatgctgaaagaggatagtttccaggagtagcgaacactgtgataatgtaggagagcaggaactgaagaaagactgttggatcttgtgaagcagtgtggtatcatggaggatgctttgattaatcctaccctgcctttatctttgatttatcctaattcatttaacctctgatgcttctaaaaagtggaagcagtgatgtaacaaatttgtgtaatttacaagcattttcacattttttatcttattctcttagaagtccttcatgcatcagggatgatagatagcccttgtcttgattgtagaagctgaggccaagaggcattaaatgttttatctatctgagggcacacagcaagtaagagccagactggaggatgctttgacggttttctgacaatgtttttgtagatccttttaatagtctcaccttcctaaactggtggttacaaggcacacatggcataatgactgtggacattctttagaaaacacaatgccctacataaatgcaaagtcatcaggctaatttagtagttgctcagttattgaacattgatcttgcaatgatttggctcttggggagttcatatctagagtatttttcagactcttcagatgtgtcagtaatttgaactgccttattaacagtggtctcctcatgcctattgtacaattcatgcctagctaataacacagatatacttttcctaaaccagatcacgtgactatttatcaaccaaatttactgaacccagggagttgttctggaattggaaactttaaattgcacttattaaagttgatgagtcagatgggactttggtgatagttctctcagacattagtaaagagtacccatcatctgctctattaaatgatcctcttttcccatagtgtttgccatgatatatgtgaagtgctttctaggaaacttaattgtgtgcctattgaattaaactctagttcgtggtttcctctctaacattgtccttggctggagtcattttaaaaaatatatcttttcttttcctgatgctttcttggtttataaaattgctgttacttgatctgtaattttgtgtgaaaacacacagtctaaattctttaatgccacttaactctataaaattctgcatgctgtttagagtcatgtttagaaccctggtctcctgattctgttttttttgagacagagtttcgctctgtcacccaggttggagtgcagcggcccgaccttggctcacttctgccttcatctcccaggttcaagtgattctcctgcctcagcctcccgagtagctgggattacaggcatgcgccaccatacccagctaatttttgtatttttagtagagacggggtttcaccatgttggtcaggctggtcttgaactcctgacctcaaatgatccacctgccttggcctcccaaagtgctgggattacaggcgtgagccactgtgcctggccccgattctgttttttgagagtatattctagttaattaattttcactgtactgttgggccactaggatacatctgttctctctcctttttgtgtgggagtgtcacagaaaaatgggcactcttgactaaggcaaggactttggagcacctaaaaccaggtgctccttcatgctgggtgtggtggctcatgcctgtaatcccagcactttgagaggccaaggcaggcggattgcctgaggtcaggagtttgagaccagcctggtcaacatggtgaaacctcatctctactaaaaatacaaaaattaaccaggcatggtgacacatgcctgtagtcccagctgctcgggaggctgaggcaggagattagtttgaacctgggaggcggaggttgcagtgagccaagattgtgccactgcactccagcctgggtgacagagtaggactccactgcagagagaaaaaaaaaagaaaaaagctccttcagatggatgggttgtattgttttatagaaaaacaatgacctgagaccttattcaggtgctttgacccatagttaagaatttaggctaatttcagtgttgccaaagattagcagttctttccaaatttacatttaatgtgggatagtaattgaactattccttcttaactcttggaaatctctggattaagcatttgttttcaggtaaaagtagacatttaattaatagccagtttacttgttgttgtatttcatattttagggtgctcaccacaaggtgtcaatgttattccttaactagcatttgaaattttatttattttacaattttgggttgagaaaagtacgggactaaaatggaatataaagcaatgttgtaaattataaataagcattcagttttaggagttaactaaaaaaatctcttagtatgattccttctaggttaaggaatacaggcttagtagatagctttctaataggcttattaggtacctttctccttttggttctgtttttttaaagtcctattgatagggaattattgaaagattataaagagaggaggagtaaatgacatagtcaggtttgcaatttagaattctaaccatggtgtcagtgtggagtggacctgagggatgagcctggaagcaatgatttctgtctaatcatttcttaatgtgtctaatcatatctaatctgttctctgactgagagattatttttagtgttaactcattttttggttgttttttcttttatactttttcaaagagataactctgttatagtaagaaaagtaataattctggtttttttctagtatagttttcattgtttttttccccttcttcttcagaaatatcctgcttttgccagtttaataagtggaagtatggtggaaaataaagggctttggtccttcacctaaattccagtcctaaaactgccatgtgtaggtagactggatgcaagactttgagcaagtaacgtcaactttttgtataccttagtctgcttttgtaaaatggggctaatatttgtctcttctattaggaatattgttagaattaaataagataaaatatttgaaagatcttaggatagttactggcatatagtagacatttaataaatgttagttttcttcttcctctttacttacctggtcttggatatagctttctcctctaagccagtattccttttccttgttaccactagcttttctacttttcagcatattttccctttctctccccagcattctagtgttttccatatctgcttttataactgcatgatggattcttcttgctcactgcccagaaaagtcaatgcattgagaacaggttttgcagcaaagaaagagtttaattattacagggccagccaaatggaaggatgggagataattctcagatccgcttcctcagaattcacaggctagagtgtttcaaggatagtttggtaggcagaggtctaggaaatggggaaacctgattagttgggttggggctaaaatcatagggggtctaagctgtcttcttgtgccaagttagttcctgtgtgggggtcataagaccaattaagccagtttcttgatatgggctacttatttgataccagctggtccatcagaattcagagtctgaaaaatacctcaagcaccagtcttagattttatgatagtgatgttatctataggagcaattgggaatgttacaaaccttgtgacctctgggtgcatgactcctgaaccataattttacaaaggtgttttcagtccctgagtaataaggaggaggttactttcaggaagggactgttatcatctttattttaaagttaagctataaactaaattcctcccgtagttagcttggcctatgcccaggaatgtacaaagacagcttgtgaggctagaagcaagatggagtcagctacatgttgattgatttctgtcactcataatttttgcaaaggtggttttactttcaaagttgcttttgttaattctaaacagtcataataaaaaatatttaacatgtgtgtgtcttttctccttaatgacattgaaatcttcttgaaggaaaggattatttcttatggctatatttctcctgtccttctatcttaccaatacttcttggttaattgttggagttcctttggcttgtgagcttactaggactcttctgtattactctttttccctaggtactctcttgtgttactaaagctacaaccaacacatctacatgttaatacctcttcaatctatatctctagctctacctctcttttggatttccagtctgcattttccaaccaccatggatatttgaacttttttgtccttgaggaacctcaaccccaaagtgtctggagccacactcatcgcgttaatactcctcttccttttcttttccctggtttgtctaatggtattatcaccatcctagtctcccaagctagtaatcttagtcatttttggtttctctaatttttcttcccttggtgatagatatagttgatgataaaccatattgattctttggcaagatgtctcatctgtcgacccttctttgtttttaatctgtattttcctagactatttccttatctcctagatgaagaaattaaggcacaaagcagttaagtaacttcaccaaggtcacacaaactagtgttagaatttgtattcaaacccaggcagtctagctccagaggctatcatataaaccagtgtatcttaaatccttacaatcttttacactgctgctggagtgatcttttaaaagatacgtgggctgggtgtggtggctcacgcctgtaatcccagcactttgggaggccgagacgggcagatcatgaggtcaggagatcgagaccatcctggctaacacagtgaaaccccttctctactaaaaatacaaaaaaaaaaaaaaaaaaaatagccaggcatggtagcgggcgcctgtagtcccagctactccagaggctaaggcaggagaatggcgtgaacccaggagcagagcttgcagtgagccgagatcgcgccgctgctctccagtctgagcaacagagcgagactccgtctcagaaaaaaaaaaaaaaaagatacatgaattttcttactcgctgggcttagaaagctttgatatatcatgtacaggagaaagttcacacttcttagtgtgatattcaagggacatttagcaatacatgcccttagggaccaaggtctttatttcaaagtcctcccactccctacttgtataatcaataactatcatttatggagtgcttactgtgttataaatagttcttcattgtctgattttccctccactcatctcaacacccccaaactgagagcacctttaggcaagaatccttgttttattagtctttgtattagccctatcgcagtgtgtatcatatatatgttaaacagataataaatgccatttctattgatcaaattgaactattgttcaggttactagaggactaaagttttgtttttcctaatggtttccatctggtaaaatgagagttgcaaaaaatttctaggctaatatttaaaaggtgatgaaaatgcaatcaggtctgtgagccttcagctgttgctttctttcagcccctgctctccacacaacatgtggtgagattttcaggattttgtgactttcaatccaactctttctataagtgttttgggaagacctttatttggctctctagaagcaaattcccctccccgcactttgctagaaagtactgtcaactataaaattccaattttaacaggaaaatgctcacaaccaataaattatcaatagcatagtgtttagagaagagatgggatgagaagtataaggtataactttgtttatcatctttaaactatggatgtgataaccaacttttaggctgcaggaagagatttttttttttcctccctgcctaatggactctaagtggtaattgcagttatacagaatacagctaagaacccaactgtaagcagactttacaaagtttatgtgctaaaactcgtgtactttcttttcttaatagagactaggctcttacccagtttctgtatgcttattttaagtaagttaggggaggaattaagaggattactgttaaaatagacacactgacagcaagaagcgaaagctgatagtttgctctgttatactctttgattctggaatatagctgagtgataaatcatattagctactgtcctacctttgtagtcactgtttggattcttaaacagccctgtctcactacttcatgtaaaaagagagaggaggagaaacagttcactgtaaggagaggccaactaaagaaaaaaacatagctagccaacttggccaaagcacatgtctgtattgcatttcataactcaagttactcacgagaattatgagtgcactgtcttagtttttaataatgctgtctccatttaacagatgaggaagcagtcttaaaatggtgagatgatttgcctgggccacaggctggttaagtgtggaagcaggtctgaagaaaggtctgccaggctccagtgcacgtgctgttttctattatgccatgttggtgccaaatcatcagtaaaatagaaaagtgtaattatgagagccttcataagaaagcaggcacatagaagatttaggaaggttttcaatcaaaatttaaaataaaataattttggtttttttttttttttttttttttttttttttttttgggacgaagtctcgctctgttgcccagcctggagtgcaatggtgtgattttggctcgctgcaacctccgcctctccagttcaagcgattcttctgcctcagcctcccaagtagctgggactacaggcacgtgccaccacgcctggctaattttttgtgtttttagtagagacggggtttcaccatgttagccaggatgatctcaatttcctgacctcgtgatccacctgccccggcctcccaaagtgctgggattacaggtgtgagccaccacgcctggctaattttggtatttaatgtgagttttggtggagcaaaatttgtatttatgtgcagcagcacattttattcttctttattcatccgtctctacatatactataaatatatctatgtatgtatacatacacataaagacacacatatacacacacacacagttttacaaaaataggatttaagcaaaatacgggagtaattatagggtgaagtcttctccttcccctttccttgtactttttggctcatcttcttctttaagcccttttcccccattccgctgccagcaaaccacccaaacacagaacataggtccgcatgacccctagtcacccttcacccatgttaacaatctgatatgtatccatctgtattttttcccatagtctctctatatgtcatagatatgatttatagatacatttatatatgttttctatataatttatggtttatgtatatggtttgaattaatttataaaattaaaattgttttataaaattggagttgatacacatacttgggtttcttgttcaatattctatgcatacattcctctaattcactttttaaatggcttaataatagtcagtggtgaggctatatcatatttattcagccactaccctattgataggcatttaatataatatttactctggctctggtttttaatgcgtgtttgttttgacagtatgcacattactgaaggaaatatttatacacatatataaccttgtttttatgatgattttgtttccatggattacattcccagcagtgggatttctgagtcaaagggtatgtattgtttcaattgttagagataatcaggttgttttcccaaagggcacaaaataaatggcattactccttaccctgattctgtgctggcaatggccatcatcaatttttatgatttttactaatttgatgagtgtaaatgatacctcagtattacttcaatttttgtcagcctggtgcaagtacttagtttgagctacaagtgaaatttaacatgttttcttaagcttattagctatttagggtattcttctgtgaatttgcctattcatttactttacctagtttttaaaaattgagttgcttgcatttttctcataagttcctaagagtacttggtatttttttcccaaatctgacaattacatcttgacttatgataccttatacaaaaagttcatatttgttgtatcgtgtaattctgttttctgttttatattttctgggcactcttgagttgaagaggactctcctataccttgcttctatagatattttttgctaactttgagatttatattattttactttttaaattaaatatttaatacaaattgcaatttattttttttttcttttaatccacccatctgcacactgatatctagtatagatagtccatatcttgtaaggtagaaattatttcttcatagatgaattcattgtgccagcacctttcttttcccactgaatttaaataatacttttgtaatatattaaaagtgcatatatactgggatctatttctggattcgctctttttttgtttatcttatccttttgtgtattttgtaactttcagattgatttaactacaggggatttttttgtgtggtctaaaatcaagtaaggcacgtcctgttgctactcttctttttgatactatttttggctattctcagaactttattcttttatgtgaactttaagcttattttatcctactttaaaagaccaatcttattggaattttaatcgaaatttttatatcagttgaagttttacaattattttgggaaagatgtcattttatattagtatacccatccaagaacatggtgtcttttcatttgtttatattttattttattccttcaacaagattgtagttctctttatatggtcatttggctttctgttacatttgttcttaagtatgttatagtttttacagtattttgtataaaataggtgttccattccattcttgtgtgcttactgctattataaggaaaagctatacttctaggaaaatcttatttatatttagtaatatatctaattgtttttattgctattgtttttttaaggaaagctctccttttttggcatacacattatcatcagtaaaaggataggcttttcttctctaatgtttatactgattttctagacttactgcatttgctaaaccttctggaactttcagaataatgttgaaaagtaataggcattgtgaccatcccagttccttgttctaattgaaaaagttcagtagtttgctgttcagaatattttctgttaattttgaaaactattggccgggcgtggtggctcatgcctgtaatcccagcactttggaaggcctaggcaggtggatcacctgaggtcaggagttcaagaccagcctggccaacatggcgaaaccccgtctctactaaaaatacaaaaattagctgggcgtggtggcacgcacctgtaattcctgaggcaggagaatcacttgaacccaggcagcggaggttgtgatgagccgagattgcaccactgcaccccagtctgggcgacaagagcgaaactacatctgaaaggaaaaaaaaaaaaagaaaaacgaacatatatatataagaaaaaaatatatatacatatattttttcatatatatatatatatatatgttcgtttttctttttctttttctttttgagacggagttttgctcttgttgcccgtgctggagtgcaatggctcgatcttgactcactgcaacctccgcctcaaaaaggaaaatattttaaataattctttttattcacttaatgcttttgttaggattggttgctgaattttttgaaatgcctttccacatttattaatattatatattttcttcctttataattgtaataaggtatgtggatttcccagtgttgtattactgactagttataggttattcttttgatcattacttataagcaagattagtttttagcagttgtgttgttgttatggggggaggtataaattaccgtctctcccaaaatcataaaattgagttgtgaatgaattgtgaagctttccatcttattctatgatcctaatctgtaaaatgatgatgataatagcatttgtctctggatcgctgggaagatgaaaggtattaatgtgaactgcttagtacagtgcctgaattgtatgtgtcagctatcattatcatcattaccatctgaaacagattaaataattttgaaagtttttagttctttgaaggttaggacttagctagtaaatcatccagttcaggtgcattttataagggtaaatcttctttcaatccctttagtactcatcgctctgtccattttttcctgcttctccttggtttattttggaatttgttttttggtaggaaaccacctatttcctctagatttccaacattattgctgaaataatgcacatagttttattttataattcttttaataatttcaataatttacagcacattttctaattaagaccagaaactactctaatgttgtagttagaaatgttgttacattatgacctcttcttggacaggatgctctctgatgttgaaattgtaataacttttttcatgtaaactaaaacaaaatatttcagatacttttgccttaaagtgtttctcagaatgagaatggagaatgtggactatatacgttcagggaatttatatctcagtttacaatgttgttcctagcatatagcaccaagctggctcagagcgaatgctcagcaaatatttgttaaatgaatgaatgaagaaatgggtgaaatataatcctgttggataataactgaaccaggtagattttttgaggaattttttaaatggtagatttttaattacctttccaaagatgattagtactaaccttggatatgttttgccacagttatagtatattttatatataaaataatttaatatttcattttggatacagtgcatgctaatgaaaaattcatgttgaaagcattgtgtttttttttgtttttgtttttttttgtgacggagtctcgctctgtcacccaggctagagtgcagtggcatgatcttggctcactgcaagctctgtctcccaggttcatgccattctcctgcctcagcctcccaagtagctgggacttacaggcgcccaccaccatgcccagctatttttttttgtatttttagtagagaaggggtttcaccgtgttagccaggatggtcttgatctcctgacctcgtgatccacccgcctcggcctcccaaagtgctgggattacaggcgggagccaccgtgcctggcctgaaagcattgtttttaaattaactgcagtttactaaatgagtaaaccagtagtcaggacttactgatagatctcatagtctttaactaggacctcttataggcaattatgctactaagttcattttctctagtttactgaatgtttaatagagaatggaaatgtaatgggtaagttaaatgagcccttcccaaaacatgttcagaacttttggcttagagcaaaagtcagctttcaaatgttgaaaaggtgaatgaagaataaaataggaagaataaaatcagcgtaaagaagtaaagcattctttgagggcagttaagtatatgtagtcttctgcccaagagatgttctctgtctctgcccctgtcctttaattttggcttgaattcacagagttactggactaagactgctttgtttgagatgtcactgcgaattaaggcttcactgtcacatctctcatagctgccttgtaacttctacattcccgctggtgccaggcatgttccaaggcagcattccaaggtttagtgatttcaccctgatgtctctgccaataaaaataaccagttaccattaaaacctaccccacccataacttgatttttacatgccgttttattaagaaaacttattgatgattttaggagaattaatacattaagtttatcaggatatcaactggcccttggagtaacttttgttctaaaagtggatgtttcaccttggaagagaatctgagactcagcatgtttatctattgagagtggactttgtaataaccttattcctacagttgattctttatgactggaaatactctgttaaaatgagaagccagattattaccttagggatttatattctagtaggacagataacagatacagcaaacaaagggtctttcattcgttgagaaactaataataatttctaaatatgttgtgttctaagaatgagtcagaatccgttccttttctacaaagggcctacacagggggtggagaaggaaaataagatttgaatattaaaaattttcacttaaatacattaatgctattaagtaaaatagcatttcagttaaatgctaaaagttagcatacatctttttattgtgttcacaatagccacactactattgcaattccaatactattacaagctcagactgaagcttgcaaaagttgagtagtgtggttcccaggtgttcagctagtaagtcatgcagagtctgagtttagtggtaaaggcctttccattatatttctccatctcatgtggaggaattccccgtgacattcttttctcttgttgagacttccttatagcacccttagtcttcccagtaaaagttttttttttttgatgatacaccaacataaaatgagccaggaattacttagagtcttgtggcacccacacaatggagactttcctaataggctttcctggagccctccagagtgtgcttaatatactcctcaccttgtgcctccccgtggaggaggtcttaatggtttactattgaggagtgtctctgactctatgaccacataggtctttttacttcttttctcccaatatgtcaagtgtgaagtgattggacccaatgattctttcttagttggcatccctgactgttcttaggcttctgttccttaaattcagactctgcaagaagtaaactaatgtcatagaggaaaatgacttttccacagtagcactgatttttctgttcccgagattctttgattctaatgacagagttaaatttgaacatgggaggaaaactgactttaaacagtttatggggagaacatctggagatttaaactcatgaaaaattcagtgtaagccaagaaattgactcagcagcattaaaaaaggagctaagatgatcttatattgcattaattaaaatatattgtccagatcaagagaagtaatagtcttcctggactttgtgttcattagatcatatttggagtattctgtttaatttgggtcccagtttactttgatgggaaagagccggggagatggccaggaagataaatgattttaaagcagtcatgtaaagactagttgggaatagaggatatttagagaatataaatatcctagagaagagaggactttggggatatagttgccgtctttaaacagttatgtagttaatttggcaaacaattttagggtaataagagtaaaagacacatttattggatatgtactatctgccagttaagtttttcacatgctttatctcatttaatccttaaagcaagtgtatggattgctgctagtcattgttaccgctgttttactcatgagatccagaaaggggaattgatttgcctaaggttgcattatggcaaaggcatttttttttttttttttgagatgaagtctcgctctgttgcccaggctgcagtgcagtggcgcaatctcgggtcactgcaacctccgcctcccaggttcaagcaattctcctgcctcagcctcccgagtagctgggactgcaggtgtgtgccactacgcccgtctaatttttatatttttagtagagctggagttttgcgatgttggccagactgctgtcttaacttctgacctggtgatccgtctgccttggcctcccaaagtgctgggattacaggtgtgagccactgcacctggccagggatgtaatttggaagtgaaaaagtatataggttgttgttattcctgagagtagaatataatgatagctatgtttattgagtgttgttatgttctaggctcttgttatatactgaaaaagaaagaaaaagtcctatcttcatggagcgtacattttaataggaaattcagaagtagataaattagttataaatttgttgctagtggtaagttctatgaagaaaaataaagtagtaggaggagaatagaggttggtagatactgaggatggtaggatgatcagggaagacctcactgagaagaaaatatttgaacaaagacttgaaaaaaattagacagcaaagttggatgggcatttggagaaagaacattccagagagaagagatagcaggtaggaatgagtttggtttgttacaagaatagctaggtggtcagtctagtttgggcatagtgagcaaagttggcaaggccagaatcatgtagagttttattttttttccaagcatgataagaagttgttggagggctttgagcaagggagtgatgtgatctgatttctagtttttaaaaaaatacctctgattagccaggcttggtggcacatgcctgtagtcccagctacttgggaggatgaggctggaggatcccttgagcccaggaatttgaggctgtagtgagctctcattgtgccactacactctagtctggccaacaaagctgagaccccatctctaaaaagaaaaaaaaaatgcctttggtgtggagaatagactgggcttcggggtcaacccaccacagctgtgatccagcaatcagaaaactgctgctggtgaaatagccatcccacacttaccagacacctaaataaggaaactgtctactgcaaaacctcactcctgaaagagcttgtcactgctgcaggaagaatggcgtctatcccttctaccttccagattttgctcaaggcattccaacatttaagaagaggatgatagaacagaggagaaggacggctaatgaggttgtggaaaatctaggacaaagaggtgtcccagaggtcaaagtaaattaaatatttctaagagagtgattaattgaatcaaatattgatggctgggacatggtggctcacgcctgtaatcccagaaatttgggaggctgaagggggaggattgcttgagcctaggggtttgagaccagcctaggtaacatagtgagaccctgcctcagaaaaaaaaaaaggttgctgattggttgagtaatgtgagggttaagaatcgaaagctgggccgggtgcggtggctcacgcttgtaatcccagcactttgggaggcccaggtgggcggatcacgaggtcaggagattgagactatcctggctaacatggtgaaaccccgtctttactaaaaatacaaaaaaaaatttgccgggcatggtggtgggcgcccgtagtcccagctacttgggaggctgaggcaggagaatgacgtgaacccgggaagcagagcttgcagtgagctgagatcatgccactgcactccagcctgggcgacagagcgagactgtctcaaaaaaaaaaaaaaaaaaaaaaaaagaattgaaagctgaatttagcaacttggagctcactggtgacatatataagagcagttttgatgtgtggtgaaagtggtacaaagaagaatggaagtgttttccaacattttaaatgattgaacaattataattatgtgactgtgagaatgtgtataaaaatagtaacaataaagaattttaccatatagtatccatatgaatcgtggagattgctgtgtgataaactttgcagttgagaattttatgacaaattaggtgcagagtttagtgatccagtaatcaagaaaaagaatgcttgttaattatttgaaataaaatgtcattagtgagaaatctttattgagaatctgttgttagtagaccttgtgttactctgtcagtcacccccctccattctgtcagtttgaatggggagctatgcacagaataaacagtagcggaatgaaaatcagagagttgttttatgagtaggtgcccaaaggaagggtgcagtcgctgaaactccctggaaaaggactgggttaaaatagggggatttaggcccaagggaacctcctacattacccctaaatctaaatcttggcctcagcgtgctcagtcatatttttattttggggtgtatgttggtgagggagagaaatgaagcccgttgtcatcttctattataaaattttacatggcgatataattctgtgtttaattattctgtatccctccatagatttgtgggggacagggactctagtttatcttgctcacggtaacagccactggtgcttagcttgttccctgtcatggggcaggtgttaaatttataaggaataattgagagtttatcaggcagtataaggaacagaagataacacctgggtaccagcatgtggaaaggtggggaatgcttgcctgacacgttaaatacattcttagagatgtagatcacattcctctgctttaggaagcattgttggatctcacttcgttcctgtacccatattgcacatagaaagtattctgtgccctggatactttgcgttattcaccttacattatcatatagatacctccttatctcctcaatactagtcacaactttttttgattgcctgtcatttgcaaatattgtgctaggtgttgtatatattttatcattctatttaattctttcagcaaccagggacattaggcaaatgaataaattgaggctcaggaaagtttaacaacttgtctaagattgtacactactttcttagcaccttccaaaagcttgaggcttcttctgttccatggtgttttttaacaggatacatacttcagtagaatttgtatgcttaatgaaagtcttttatactttttttaagatcttaccacaattcctttcttttttgttgattatttgatcaaagggccatgtaattctaacacttcttgaaacattggagctgaaaagtaagactggtgaaatagctgactttaaatattgcgttttcaaggctaaaaatgagtgtctaacttactgattcattgtatgataaactaatattatagaactgtttttatatagagcagacagaaaaagttttataattgctctgagaaaaaaatatacttactcaaaatgtgttaagcattccaattttgatgcttcaacacggagctgtcccctgtagccttggagaccataacttcacagtgcttgtttttaaagtaaggttttcttttgaatgtaagcagattttgatggtagaaagaagttgtcgtgtggaattattctgtggagccatttgaatagacttgaaacattgtttatgcataaataaattcaccactctgaaaagtattctgacaaggcacggcatgtgttggcatctgcaagtcagcctgtactgagttttcactacccacaagaggtggtgaaaacgatctctggtataaagcaaatggaaaaggtaaagtgtcgggattttctttctttctttctttctttctttctttctttctttctttctttctttctttctttccatttttaatttcttttaacctttcctgggtattcatcaaaagtttcttttcaaaattaattttttggtgagatacagttagcatgacagaactataaatgttgatgttcattttatgtttttttgggagtttgtttctctcttcttttcctttttttaatgctgttttgcaaataatttgtgtgatcatttttttcactatggaccatcattcatgtgttgttttcattcagcagatacttattgctcttctagttattatactgttatgtgctggagatacaaatgtcacgttatgacaccagccttccaggagattttagtctagtgtgttagtcatacaactatgcacacagacacagtacagtatattaaatgcttggatagtgtctcatgcttgcctgttgggaacacagggaaaagagaccccaaggtagtcatgggaaacttcctggagaaagtgataccatctttccttttgttccccaaacaaacttagcccccaattgcacctggcatattttaaaaaattaaagtgaaatctgcatacaatttttaaaaaatcaaattctaaaatgaaaagtaaaagttctcgttcaaccctcccaaccttgacccagtcacagcccttagtaataaccacctttcacactttctattttcccttatgatggttatcattaaaacatgaaacaatgtatttgtttctgttatttaatacaaaaacattaaatagtgatttggctttggacaatgaaagatgaggaatttaccacatttataatacctgccaccttctctctctcactctacctctgtgtttttgttagctatctcatttttacattatcaagtttaaaaaaactttacatgttatgttgttataatcagctcctgtgctttgtctatagggtgactttaggagttgagccacagacaaatacccttcataatattattataggcagaagtagtgaaatgtgctaacactggaagaatttcccaagaatcaaggtccagtggtccaatggatggtttgtcttttttttctttttaccacaaagacctgacttttttctcattggatatgtgaattccaaccttctttttgaatgacatttagtatggtgcttgaaaattcaaacagaataatgcctcatttatttggcaatatcaatttataacagccaaacagtaatccaaagttagcattaaaaatccgctaagcaaattggaaaggaagaactcaaattatctttgtttgcagattatatgatctttatttatttatttatttatttatttatttatttattgagatggagtctcactctgtcatcaggctggagtgcagtggtgcgatcttggctcactgcaacctctgcctcccgagttcaagtgattctcctgcctcagcctcccgaatagctgggactacaggcgtgcatcaccatgcccagctaattttttgtatttttagcagggatggggttacaccatgttggcaaggatggtctcgatctcctgagctcatgatctgcccgcctcggcctcccaaagtgctgggattacaggcgtgaaccactgtgcctggcccagatgatatgatcttatgtttggaaaaatcctcaagactcaaccaaaaaactgttagaactgataaacacattcattaaagttgcaggatacaaaataaacatataccaaaatcagtttctatatgccaacatgaacaatctgaaaaagaaatcaagaaagtaatcccattcacaatagctacaaataaagttaaatatctaggaattaactaaagacatgaaagctctgcagtgaaaactacaagacattaatacaagaaattgaagcagacacacaaaaatggaaagatattccatgttcatagatttagaagaattaatattgttgaaatgtctgttctacccaaggcaacctgcaagtttaatgcaatttctatcaaaatatcaatgacattcttcacagaaatagaaaaaagaaaactcctaaaatttatatggaaccacaaaacacccagaatagccaaagctatcctgagcaaaaataacaaaacttcaggaatcacattacctgactttaaattatactacagatctgtagtaaacaaaacagtatggtactggcctaaaagcagacacatagaccagtggaacagaataaagaacgcagaaataaatccatacatctacagttaactcatttttgacaaaggtgttaagaacatacattggagaaagatcagtctcatcaataaatgatgctgggaaaactagatatccaaatgcagaagaaggaaactagaccactgtctcttgccatataaaaaattaaatcaaagtgggttaaagacatgaatctaagacctaaaactatgaaactattaaaagaaaatattggggaactctccagaacattggagtgggcaacagttttttgagcaatatcccacaagcacaggcaaccaaagcaaaaatgggcaaatgggatcacatcaaattaagaagcttctgcacagcaaacaatcaataaagtgaaggcacaacctagagaatgggagaaaacatttgcaaactacccatctgacaagggattaataaccagaatgtataaggagctcaaacaactctgggaataaaatctaataatccgatttaaaaagagcataagatctgaatagacatttctcaaaggaagacatatacatggcagacaggcctatgaaaaggtgctcaacatcactgatcatcagagaaatgcagatcaaaactacaatgagatattatctcacgctagttaaactggcttttatccaaaagataggcaataacaaatactggtgaggaggtggagaaaagggaacccttgtacactgttagtggaaatgtaaattagtacaaccactatggagaatagtttggaggttcctcaaaaaaactaaaaactgagctattacatgatgtagcaatcccactgctgggcatatagccaaaagaaaggaaatcagtttatcaaagagatacctgtactcccatgtttatcacaatagccaagatttgggagcaacctaagtgtccattagcagatgaatggataaagaaaatgtgatacatatacacaatggaatactattccgccattaaaaaaatgagatcctgtcatctataacaacatggatggaactggaggtcataatgttatgtgaaataagtcaagcacagcaagacaaacatcacatgttctcacttatttgtgggagctaaaaattaaaacaattgaagtcatggagatagagagcaagaagtatggttaccagaggctgggaagggtagtaaaataggaggtggtggggaggaagtgaggatggttaatgggtaccaaaaaatagtagaaagaaagaataatacctaatacttgctagtaaaaacaagatgactatagtaaaaaaaatttaattctacatttaaaaataactaaaagtataattggattgtttataacacaaaggataaatacttgaggtgatggataccccatttaccctgtaattattattacttattgtatgcctgtatccaaatttatcatgtagtctgcaaacatatacacttactgtgtgcccactaaaattaagaataaaaaatttaaacaaataataactccctagttacaaaaaaatccactgagcagcaaaaacatctcatacctaaaatgttctacattttatgcctagtaagaatccataggcctcattcagaagctgtttactcttattttgtgtgtcattctacaaatcatgatctagtctccaagtctgtaacagattaaatgttataatttagtgatgctacaggcaggcacattgaaagccctaagaaatgtcatatgggaatctcatagagaagaaaggcagtctgtgcatatcatcctgaatgtgcctgatgttgtctgatctcagaggctaagcagggttgggcctggctagtacttagatgggagaggaaaggcagacaaaatattaaatgctgactatacaatttaaaaggtattatgatgtagggctgtatgatactgggcataatgatgctctagtgatcatcaacctttacaaaaagaatcactattttcattgtacagtgtcagaagacagggaagtaagtagtcattgaggatatattcctatccaaaatggttaaattttgaaatattgttcatacaattaaaaaagataagcgtatgatacagttatctggaaagttaatttgtgtgggagctcttctccaaggtcagtggaattgtatatttattatcacatttgtgacatgaatgggaattgctttattagttattaaaaactcaacaaattagctattgtgtgaattattactgggaggcagctgttgactttgttgtgaaatatgtcagctaggaaaaaaggcaatctgtctctctttctctctctctctctctctgctatatcagtggattgaccattatttccatggattaaaagattggacaggctgccactggggataaagggaatatgcttccttgtggaagagtcttggtttgggatactgctaccttaatactccctgcctttatttcttgagtattttatgtctttttccttggaaatatttccaaacttgaagaaacattacagaaatagtataaagagctttccccctgctaaccatttgaaagtaaattagaaagatgatccctcagtactcccaaaataaatcatggatatttgctacaaatacgaacatccttctagataaccatagtatgcccatgaaaaccaggacattaaccttaacatgttactggcatctcattcatccatccattcaagtttcttcagctgtctcagtgtcctttataactaaaggattccatctaagaccacaaattacatttacttgtcatgtatctttactctctgtcaatctgaaataatttctcagtctttccttaagatttttgaagagttttaggccagttaatttgtaatgtttatcaattttggtttgtctgatgtttcctggtgaagagattcaagttatgcatctttggaagaagtaatgcggaagcaatattgtattgtttttgcatcctgtcagaggggacacattttgatttgtcctattgctagtgatataaactttgaagccttaattagtgtctaccatgtttcttctctgtgaagttaaccttttagactttgcaatcagtaagtatttgtggggagatacatttttagatgataaatgtctcattttttaatcaaatctctgtccactagttttagcacatattgatgtttcttggctaaattaatttttttaaatgatagttgccaaatggaatggttaattcagttctcctacatttattagtgtgacattccatttaaggaaaagctttcagctttttctgtttaattgtatcaatgtagacacatggattcctgttttatccggtgaattattatctggtagtataagtatttattttgatactcaaatcactccagatttggccatgggaatctcttcaatctggcttctgtgttcttaagacatatttccatcactctttgagtatgtccttactttctggcataatatattacaggctcatattccctttgcccctggattagaattagtcatttctctaaggagttttgttttgttttagtacagaataataataataaaaaaacccactgttgattgacttaaacatttctgttttttagagacaaggtcttgctgtgtctcccaggctggcgtgcaatggcctgatcacagctcctcaaactcaagggattctctcatctcagcctcttgagtagctgggactacaggcttaggccaccatacctggctgatttaaaattttttagagatggggtcttgctatgttgcccaggctggtcttaaagtcctggtctcaagtgatcttctgttctcagcctcctgagtagctgacaggccaccaagctccagttaattaacttttaaagcaatttaaataataagaaaagtttttaggctgggcgcggtggctcacgcctgtaatcccagcactttggaaggccaaggtgggcggatcctgaggtcaggagtttgagaccagcctggccaacatggtgaaaccctgtctctactaaaaatacaaaaattaactgggcatggtggcgggcgcttgtaatcccagatgaacctgagaggcagaggttgcagtgagccaagatcacgccactgcactccagcctcagcgacagagtgagactctgccaatatatttaccatttctggtgctcttcatttcatttattttcgtctggtatcattttccttatgtctgaagtatttccttttgcatttctcatggtgcctttctactggtaattgaactctttcagtatgtctgaaaacatctctatatcatcttagtttttggaagatgctttcattgggcttggaaatctaggttacgggttttttatgtcttttaattctttaaagatgttgctccactgttttctagcttgtattgtttccagtgagaaatctgtcattctagtatcctctcctttatgtagtgtgtctttttttccttgggctgctttgtcactgttgcaaggcaatttgattctgtgtaccttggcatagttttctttgtgtttttcatacctggtatttgttggtattcttgcatctgtgggtttgtaatattcatcacatttggatattttgcaaccattaattattcaagtattattttttctgtctcctccttcattattttccccttcatgaactccatctaaatgtatattgggcctcagaagttgtgctatcacacactgatgccttttattttgtttttgttttagtcttcctctgtttttcattttaaatagtttctacccaaatgttcactgacatttagtatatcatcaggtttactgtttttcttgtaataactaatttgttcatccatcctttaaaaatatgtgtgtgtatatatatgtatatgtatatacacacacacacacatatatatatttttctcatttcagacatcatgatttccttttttttttgcagtcagctttctcagattaaacagacattgtgattttcatctctgaaagttcaatttgggtttttcaaaaattttcaatgtcactatatgttcagtctattttctggcttttaaacatataaattatagttgtaataactgttttaatggccctatctactgattctgtcatctctagcaattttggatcagttctgattgattttgctgctcattttggattgtattttcctacttctctgcatatacagtcatttttaattggatgtcaggcattgtcaattttaccttattgtttgttggatatttttgtgttcctataaatattctggaaacacagttatgttatttggaaaaattttcttcctttcagttcttgcttttacgcttcctcagcctgtactagagcagcatcaagtccaggattaattttacctacccctgaagcaaacctcttctgagtactttattgcgtggcccgtcagttatgggccatactctggctgtcaggaataggccctgttcctggtcctgtgaaatctctgctttttgtttcctctgtctaatccttttagattgtccttttcctggccttggatcatttcctcagatgattcactgatcagtactcagctgaatatttgagtgtgaccctctgcagatctctggagttctctgtgtgcagttctgtcttcttcagtactgtgccctgagaactctcgcactcatggcttccctggactcctaaatctttcttctacactcaggtagaccaccttattctgttgggatccccatccctactctgaagcctggaaactttctctaagcattaagccggggcaattgtagaactctcctcatgtgtttctctactctcagatgttactacaagttgtctggtggccagtcttgaaatctatcctctcatgttttcctagggtttttttttttaagttatttcaggtgggattaaatgcagtctctgtcactctattttggccagatgcggaataaattttgctggttcttaacattttgatgtcctaaagatcattttagtatcttcagggtaaaaaatgaattttctctacactgtagtgtaaggtgtggaccataccttgctacctctgtatcattttctttgtcctctttgttcttgttgaaacttgaaccaatcttatgggaagctgactatgggccaggcagagtagaatgggagttagattcatatgatctgattccttcccatagagaactcagagcttagagttacataatatcctctaaatcatagctcttaaattggtagaactgagttttgaaccatatattttggccttcccgttgaatgtctttttcactatagcagtgttcttgaataacccccatacatatacatacatagactcacacatatgcatacatacacacacacatacacaccctttgtaactcaggaagcctcatgagcatatggtctaaagtgttcacccccttcagattgctttttgcttatcagtgattaaaaggcttgggaggcatttgagaatgtgtgaggctggtaatgaactggaggtccaacgggatgaaaagggaggaaaaaggagagtgtggtgtcttattttttggaacagacttcaataattataattactagtatgctggtataaacttgcctttggagacactgtggtaattttgatttatgtccagttaaaactgcaacaaaactgccaatacagaaggaattctgtgtttactcattttcttcagatagtaggccaaatggagaaattcgttaagccaatcctgaaacactataccaaaaacttaaatgagcaaaattaatcacatgtattggcaataggggattccatgaaaatgtttcaacttctggcagcaacttttaaactttctaatttcagtaataaggctatttgcataactctaaatgtagcaggcaaatggtacaaagaagaaagaagacagtgtggtgtagagaaagaagggaactggaaattaagaaacttggcctttctaaactggcatagctttgagacattggacaagtcattgaatcttaactgtggtttcattttcatatttaaaacgagagcattgaattaaaagacttctaaaataactcctagtactaacattatcatggtcctttgactataaaggtgaccttaattgacttaagagtcagttttctagttatttctaaaactaataacattttaaaaaaatgatttactatactatcacattttctattctacttaaaaggtcattgtccagtttgattgccttgtaattcacaaaaatttagcccagattaattttaatgattcctaaaataccaagtcctctagcaaaggtggagatttgccattatagaggatatccagtgatggtaccacaagttttgtggagaactccccaagtgctttggacagtggcattcttgctggaataaatatgtaggttgtaaggggatagtaataattagagtcactgctaatatttgagtacttatcaagtgataaaactgtagatgcttttgaagtatttagttatcagaataaacctttgaaaaaatgtatgatttttatccccattttgcagatgaagcacgggacttttggttaatggttaataagccactgagctgggatttgaaccatcactggcaccacagcctaccttcctaaccactacactatgctaactactatatttataaaagtcaaatgtatcaaattgcctgatgtatttccacaagtagacatcattaattgtgaaaattctgccttcaaaaaaagtcctttgacattactactaaattatctgtcattctgaactgtttgttaccattctgcaaaaaaaaaaatgaggcagttttgccaaaatgttttcagcaatatccttaataagcacattgttgagttcagctgacattttgtaagcaagattttctcagtgaaggaagtagtcattcattgacttacactcaagagcaaactgcataaaaatgcccttctcactttgcttttatgagctacccaaagtctgtgtaaacccaagaatatagatgaaaccaactgtttctgttcagatttttgttgttgtctctacttctccatatctttattattaatttccctggaaattagaacttttctaaaactgtaagatatttaaaaatcttaaaatgtgttttcctttctaaaaagccttctttaaagttactcattactacaggttgagcatcccttagccaaaatgcttgggactagaagtttctcaggtttttttgtttgtttctttttggaatatttgcattatacttactggttgagcgtctgaaatcccaaatgctccagtgagcatttcctttgaaggaaatgttgatccacttgtttatgctcaaaaagttttgaattttggagcatttttgaattttggattttcaggctagggatggtcaacctgtatcaccatatctgacattcccccctgcaacctgatattatttcctttggtgcagagtttcagaatcaaattaaggaaatgtcatgtttccacagatgagttctctgaaaaacttcctcttacagtgaaagtcatagtccaggagaactttagatgtggaacttcagggccaggtggaaggccattatgaactcttgcattggctctcttatgatcataagcctgaaagtacagaatcctggaaaaggaggcctctctctgcacccatgtaatttttaaaaaggagagaaagatatgaagggtgctaaagcttgatcctaaaagatcagggcaatgaaaacaaatgataatgaataactagtactagaattaaaaatgtaaggcattttaaagagatggggtgccagttataattcagttttgaatactacattgcaaaagaactatttttgaaaatagttaagggaaaaaatatctgaattgttgaataagttccccctatttattcctggtaaacttcaatcccatcttactgcttcattctaagttcttgtggagtgtgaatatcagcaattttctgaccttcaaagaggaacttgaataatttcattggaactctgtgcgttttttgatagtatgattctttgagtttcttgggatgtgttgtcatttttaccttgcagtgaacctcactattgtcctgtctaatgactttgaataaaatatatattttttctttcttgtccctgaagtccatggtggaagcttttgcctcttgtttagacatgatggtggtggcttttctttcggatctccacaaacagcagattcacagcgggagagtctttctctctgagacttacaaatccatgttttttcttcacagctcaacaccactgctttctgttaagatgtaaacaagtctaataatttctttttcaaatgccgttctgatgaaaacctgagtagttaaaaaatactatttaaattacaatttaaaaattgtacaattattaagtgtacattaaacacttgctaagtgtacagctgtttatcagacacaatacaggaggggatacaaaggaagaagacaagaagcgaattgtctagttgaataagatttagcttgtggaagaattagtgtcaagataaatgtattcaagtgctgaacttcatggtacatatgcttactgaataaagcagcatgaaaggtcctttgtatccttagtttatgagccttaaaacagatttagaaaaaggaaaagccaactagacacagtggtgtgcacctatagtcccagctactcgggaggctgagacaggaggctcacttgagcccaggagtttgaggttacagtgagctatgatcataccactgcactccaatctgggtgacagagcgagatcctgtctctaaaaaataaaataaaaaaagtcccacctcccaatactatggcaattaatttcaacatgagtttgggagggtacaaatatttatactatagcacctataaaatcatcatggcctacaccctcccacccccactttggtagcgtttttagtttctttgtagttatcgatctattcaggttttcaacctcttattgagacaatttggtaatttatagtttgatagaaaatcattcacttttcctgtttttttttttttttaaatttattggcctaaagttgtatataatattccttttaaattgtaaaaatcttgtccatatctgtagtttgttttgtttattgtttctaattctgaagtgcttttctgtatacttcctcaagtgtgtttattgtgtaatgaatacattttaaattaaacttactaaaatgcacatggatagtattatgaaaaaatttggaatgatttagcttgcttttatgccaaatgtggttcttcttgcctgaaatgagttagttgagatgtaaattggaaatgcgaagagttgattattgttctataactgtcgcttcatcagtgttgttagtttttgagggtatgcatatcctggttagacttgatgcactaattatcacatgctttcacaccacctaccatctcctcaatagaactttcccacccacgatgctatttacttttccatgaaagtaggttaattttataagtatttataagcaatagtattgtcttatttgatgccattaccatgagattgtgttcttcccaactatgtgcatggtctagacagaggaatgtagaagcaggcttttgggtagactcctggtcttttgggaaactatcatatttaataaatccatcaacgagtatttgagtgattcccatgtgccttagactgctgtaggtgctgtagaggtgcagagaatttattaggcatttaaagtctatagcactgtagctctgatcaacctttccagcctgcctctcatgatattgctcttgtgccctctttgttttatagcacacaacccactttctgttctgaacgtgtaatgcactctgcttccttaatgactttgaaaagctattcccaataccctaaacagcagttcccttgcatacagctgtcagagtcttagctacccttccttcatattggagcttaaatgctgtctcctttaatgagcctttcccaagaacaagttatcttagactaatgtatccattttgcaagggtacttagatttgtagatacagataacttcaataatgtgtttgataaggtatgtcaggatagctttgggagcaagatgaagaggtgtaagctaaatgacagaaaagataggtcgactggtaactacttgatgacttgctatctagagggaaatttttaaatgcatggcatagggctctgctattgacctagtctttttcaacatgtccattcatttgattacttacctgccaaatgtttattcagtactgcatgctggaaactatgcaatatgctgggtatacagtggtgatcaagggagacatggtttttcctcttgagaataccacaatctaagcctttgaacaaagatatagaaaattgactaagttaatgcagtggtaacgtggaacttatagagacagtgattattgcgtaacggaattagtaaataagatctctggggactggaaagatagaattatattcagctcatcttaaattttaagatagagtttaattgtgaaaagaatatagtcttatacttgagtcctaaaaccaactgtatcagaagagggaggcttggcatatgggcaacagtctgagtaaaaaaagatacagaacctttagtcagtagtcaagtcaatatcaataatcagtgagatattactgccagaaaatgttgggttattaaattgatagacatgtagtgtctacaatgagaaatgatcagtcttttctggtttacaaacattaaacaatgttctcaaggcagtttcccaggagtttttctacgcttgatatacatctaaaaaaataaattaattatcactgaataaatattgcatagcatctgagaaaaatagaatcagagaaatcttaaaataatcatttttaattccgtgggtgtctgttgtgctttggggggttaagagaagggtgagatgtgcccctctgtatttctgaggaaggaaagggagtaatctttattacagtctgttactgaaggcctctatatacaaagtactacggggaagctcaggagcacagacactaccaagttataggcctaggttttcaaggagtgtggaatccactgctggagaaagccggaaaacaaatgattatagatgatatgaagtctagatggagcatgctcccagtgctgttgagggagaccacaacccttaccaaatattgactattcctgggctcctacttgagttcttcctcttccaaaatgaatttgtgggtctgttttgtacctacaggtgcctggtctttttgcattgcagcctacatacatgacatctcgtggtcaggcctccaaatgacttatttatttgccttgagagtaaagctccttatcctaatataccaggttttcataatctgacctctgcctgcctttttacctcatctcttgccatcctctcccatatgaccctgtgctccagccttgctaagctgcttacagttcctgaaaatgttagggtaccttttttttgtttttgtacttgtttttgcttttgtctggaatatctttctccctttttatctagctaattcctacttttccttcaaatgttggctcagtgtttaccccctccaagaagacttttgcttccacttgaccccgtccctagaatagatgctccttttttatgttcagcccttacccgatgcatgcctgtgttgtagcctgtataatgctgttatgattgtttacaaaccaagttgtctacaaagtttgagcattatgaagataatgctctgatctctgtatttttattacttaacagagggcatggtgaacatttaggccttcaataaatgtttattgcatgaatatgtaaataaatggtatatttttagagagcacagactgtctgcaatgttttttctcttaacactagactcagtattagcaacatagaaagtgctcaatgtgaagacaggatgaatgaataaatttaaagatacgttaacatgatcagataaggaattaggctaaagatttataatgtgcaggtgttattttgactaatgtgataatgtgctcagtaaatggaatcatgaagagatggctgtgaaagaaggataattaatatttggattgaaagaatgtattagtttttgattgctggataataaattaccacaaatttagtggcttaaaataatacctgttttattgtttctcggtttcttctttaggccggaaatttgggaacagctgacttggttctctgcttatggtctcatgaggctgaaatcagtgtcagctgcgctgtgttcttttctggagctcagggtccttgtttaagctcccatggtttttggcagagttcacttccttgtggtttataggactcagcctcttgttctcttgctgatagtggtctgagggctgctgtcagccccattgaggccacctgcagtttcttgccatgtggcccactttataggccctctcatgcttcaaatctcttctttcaggaagggccagtcccttttaagggcttacctggttagagcagacctaccgtctgtattagtcagcttagactgccataacaaaataccagtctgggtggttttaacaacagacatttattttctcagagatctgaaggctggaactctgagatcaaggtgccagcatggttcagtttctggcgaggctctctccctagcatacagatggccacctgcctgttgtgacttcacatagcctttccttcttgcatgcgtgtggagagggagagatctttttctcttcctgttcttttgaggccaccctatgacctcatcaactttaattacctcctgaaagtcttgtttccaaatatagtcacattaagggttaggatacttcaacatgaatttggggcgggggatgaagggatacagttcagcctatagcacccagctaatctcccttttggttaactcaaagtgaactgatacgtgaccttaattatagcccctcaattccttcaccttgtaaagtaacatttctgggagtaatatcctatggtgtttataagtcctgctaataaccaagagaaggggattatgcagagaatgggaatcttggggtgacattttagaattctgcctaccccaaagatagtgggataaaatgcagcagcagatttagttgagatacgagttatattatgaatagaatcgtagaataatggaagagaaaagcacacagcaacctgatatataaggggaggaatactgagcttggacacatatatttggaaggtttaacctaaataatgtgtctgtgaaaaagcactttggaaactaaaattatttaacatgtaaggtgttactgatgccaaggtttgtggggagatgatgacaagattagcttaggaacgatgggtttgtgggtgggaaataagcagtgaagaccacgggctttggagttaaacaaacctgggtttaaatagtgattctgccacttaccagcttcagttttctcacaggtagaacgggaatgattacgtctatggatttttgtgagaattaaatgaaatatccatttcaagtttttagcttagtgcctggcacatagccagcactaaataaatatgtattcattcaccaacgaattattaaataaatatattctatgtgccaggcactgtattaggcctcaggtttctggtaaatattaatactattgtgcctgagctagatttcattggactttgaattgccaggctaaaacaacagagtttaaatgtttatttaaatcgccctgagctgttttcaagcaaagaataacatctaatattagctttttagaatttgtgattatttggtgctagtacatggtggtttttttcttttgtataaagagctcaaatttgcctgtagtcattctaattattgatacactaagacttaaaagatctccatacctcttacatcattgttttgattttttgaaggacgctattgaaatctgtgttttaggagagcattctcatggcagtgttccaaatggattgaaaagggcagagattaaaaaggaaggccttttattaacgcagaaccagggtgcttacagtgggaatgatgaaaaggaaattatgcggataagagcattgggaaagaagaatcaataggtttaggtgattgaacacaggaagtatgagtgagggaaaaggcagcactaatataatgtgatgtcagttctggagcactgcagtgattcatgactatcatggagcggatttatttgataaaatttatttgaataggtggatttatttgaatagaattctttaaaaaatggaaaattcatttagtgtagaaaaatgtagatcacgcctttttagacaccagaaaaatccttttatcatcgatggaagatgttatcaacatgactagaagtacatatatgttaattacccatagatacataatgaaaggaactgtgtaggcaaccacacactagctattagagggcctagagtatctaaaaggttagtgtccatcagagaagaagaaagataaagtggtgaggtactagatgcattagcacgaatctttgaaattgtaagtttatggattgtgatatttctagttgtctctaaggaaatataattttagatgtgtataaggcaggcgttctggatgctgttggggaagataaactacaattagtaaccgagcaaatgaagaacagggattctttggtactaagaacagaatagaattgagaagaataagagttgaagctaagaaaccagttaggggctattggctaggtggcagtctatctggggtgaggtgaacggatttgaaatggctttaattaagtcgttgctgctaaatatgtagaaactgtctaggccttaggttgcttgatttttgtgacagtggtcattcctcccttgatgggctgttcctttaactgctgtcataccagctctcccagctgtccttctgcttctccctggatattctggttcacaattttttgcaggacttttttcccccctaatttcttaagtattgatattcctctggattctgttctaggttctgtttactctcattctcgcagtcttcagggtgatctcattcactttcatagcgttcataaccatctgtgtgctaatgactgccacatttttatctctggccaagatctgtctcctgagcaacagatctttatattcaactcatattcaacacatcctcttggatgttacacaggcacctccaactgaaaatagaactttcttctcaaactgttgctgccgtgttctacatcgcagtaaatagcatcaccattttttctggttccccaagccatataccagcaaacattcttttctttttctttggcagtgtaatttatatacaataaaatacatatatatatatatatatatatacacacacacacacacatatacacatatatatatacacacatatgtatacatatatacacatatatatacacacacacacatatacacacagtttgatgaattttaataaatatacacacctgtattgccattgttaaatcaaggtatagagtattttcattatccagaatgttccctcatttgccagagttctgacaccctcattcccatccctgggacacttttcttactgccttatcatttattagctttccctgtttttgaacttcttataagtggagtcatacggggcactcttttggtttctggcttcttttgtttaacatacaatttttggaatttatccatgttcttgtatagattagcagtgtattcctttttactggtgaagcagtatttccttctatgaatatattacagtttgcttattcattattctgttgatggatatttggattgtttctactttttggctattataaataaagccgataggaacaccttacacaagttttttgtggtcatgtgctttctcctatttatttatttatttgatattttagagacagggactcattctgtcaccaggctcggagtgcagtagtgcagtcatagctcactgtaacctcaagctcctgaactcaagggattctcccttcagcctcccacgtagctgggaatgggtgcgcactaccatgcttggctaatttaatttttttagcgacagcgtcttgctgtgttgctgggggctgatctcaaactcctgatgtcaagtgatttctcccatctcagcttcccaaagtgttgggattacaggcatgagccaccgcacccagcccacattatacctgagagtgaaactgttggcttatacgagaggtgtatatttaacttattaggtattgccacacaggtttccaaagtagttatacatttttacagtccctccagcaatgtttgaaaagttccagttgctctacctcttgccatcatttggtattgccagttttgtaagttctagccattataataggcttgtagtagtgtatcattttgattttgatttttacttttataatgactaaagttatgagcaccatttcatgtgctaatagctcattcttgtatcttattttttgaagtgtttgttcaaatattttgcctattctcaattattgggttgtttatatttttatttttatgtaggaattctttgtgtattttggatagaagtcctttgtcaggtgtacacacacacacacacacacacacacacacacacacacacatatcccaagtgtttttttctcagtcatgacttgattttttttcatttcctcaataatgcccttaggactaatttttgcctagtcagagtttttgcatatattttcctatgtgttattccagaggcactgtagtcttagcttttacatttaggtccctaatccatctcaaattaatctgtgtgtatgaggtgaggtacaggttaaggttcatttatttcctatatacgtattaagttatttcactgccatttgttgacaaagtttttcttttccttattaaaaaattgagtatgtatgaatgattctttctataatctgaaatgtgtgtgtgtgtgtgtgtgtgtgtgtgtgtgtgtatatattttttgtttgtttgtttgtttgtttgtttttttgagatggagtttcgctcttgttgcctaggctggagtacaatggtgcgatctcagctcaccacaacctctgcctcccaggttcaagcaattcttctgcctcagcctcccaagtagctgggattacaggcatgtgccatcacgcctggctaattttgtatttttagtagacacagtgtttctcaatgttagtcaggctggtctcgaactcccagcctcaggtgatctgcccacctcggcctctcaaagttctgggattacaggcgtgaaccaccgcacccagccctaaattatatttcattgatctgtttgtttagttctgtgtaactactgctgccttacttactgtagctttatgataggtcataaaatctagtagtgtaagccctccagctatttcccgcccccaacattttcttggtgttctaggtcttttgtatttgcatatcaattttaaaagcagtttgccaatttctattaaacaaaaatcttttgggtttttgattgggattgctttggctctagatcaatttagggagacttgactgcttaataatatcgtatcttccaatccatgaaatgatatatgtctccatttatttaaatattctttaatatctcagcggtgttttatggttttcagtgtacaggtctttcacatctttctttagatttattcctagttatttattgatattaaatagaaacactgattgatttttaatatattaatttaaactcttgcaatttgccgaatttactcattagttctagtagttggattgtagatttctttggatttttctacatacacaatcatagcacctttaagtagatttgcttcttcctttcctatacctatggcttttatttacttttaaaatgcattattacaatggtttggaccttgagtatatgtggaatagagtgatgaaagtgggtatctttgccttgtttttgattgtaagtataggattagctgtagatttttttctataaaccttttatcagattgaggaagctcccctttcctcctagtttgttgagggttttttttttatttttaaccattaacaggtattaaattttatcaaatgcttctttttttgaatcttttaagataattatgatcttttttcttcattctgtcattgattgattttcaaatattataccaaccttgtatagtatttgaaaatcagtcagtttaacagaatggtataacctactcagataaacccacttgatccagttgctttatccttttcatatattactagagttgatttgctaacattttattaatggtttttgcatttatgttcatgtataataattggtctataatttttttttcctatagtgttcgtgtcatgttttggtatcagggtcatgttgacctcatgaaacaaactgaaagcatcccttcttcttcttggaaagatttttgatgctactggcattttaccggtgaagctatctgagcctagagtttcctatgtgagaaaatttttaattgcaaatataatttatttaacagatggtggttctttagattttctgtttcttttgtttgttttggaaatttgtggattttaaacaatttgactatttcatttaagttgaatttattggcatacagttgctcataatttctccttaattccttttaactatgtgaaggatctgtagtgatgtcttctctttcatttttatgctggtaatttgttttcaagcatcattctttatctctcttctctctcacttttcacatttacaccatctttcctgtcactattttaatttaggacagagccatttgttacttggactgttggaataacctcacactaaccttcctgtttgcagtctcttttctctgtgtgccattcacctattacaagaagaaatatctttctaaagtgcaaatgtaattaaatctttcccagcttaaaactttttaatggttttctgatgccttcagaataaaaatctaagcaccttcatatgaaatacaaaaccattaattataaggtattgatttgtttcatagacatctgtctttcctgtatttcttctccctttttatctctctctgcaagccgtagggaactatacttcactgcctttgtaggtattctaatttgtcattctttgtcttaaacttgaatgcagggtttctgatgtcatatgatcaatattatcttctaacttctccttcagtgagattatttttaatatctcattggcttcatctcagttaatctttattaataatttacttgacataattttttttattcaatgtatctttacgttcacagaaatatatagcctcaaagcactttagtccagtttcagtttatctcaaatcatgttaaaaaataggaatgcatctctttcctatcaccagaatacctatcggatagctatttgatctctccttcccaacaccagtgacgataagttttatttcagcttgaggatgttagtgttgatattgaccactcacccatattactacatgatatatatttctttctttatagattcttttgatagtttgcaaaatcaagaaacattactctgaaattggccaaccagtttggaggtaaagcagttgtttcttttttagttagtccttcacctctttatccccaggggaatcaggatgtcccatgttatttttcaaagtctaaatacttcaaacttttattctcctgttgggtttgaaaggggggtgttattagctctgtcttgatgcagcaggggctctgaaagtctatttggataacactgaagattggaggaaatgtgactctgtctgtgtgttttaagcccctaaggtaagatcagaaagcgtctggggcagtgagagccaaatagttgaagtcatacattaattatggtcatgaatcatttttatttttaaagtctttaccatttgacaaggttgtttgcatagtaccagtgaagttttaggcggccctttcctacttttaaagtaacaggctattttgttgatttcttctctgggttgatgatgcctatcatttttagaattctgataacatttttagtaaaaattgctgtttcctcacttgaatgagtggtctctatgtgatttttattttgatacactattttgaagttacaaatctaggaaactaccacactgatcttccaattaatctcccactgatgtcaaaaatgtcaaattttgggactgtgcttttatgttcagacattaggcattaagccagcaaatacattttgagtccctcctttgtgccacattctgtgcttagcatttggaatacaccttagaagatgaaatttatttttcccccagaaagatctggtgaaataggcaaagaaataaacagttgctgtaaaatagggtcatgtaatatttttgtggtgccatacacatgaagtagcagatttccagaggaaggagcacctctacctgtccgtaatagtcaggaaaggcttttgaagaagaggttgcttgagctgcctcttgatggattaggagtttatttatctgttaagggatgggaagaacttgtaggcacatagaacaactcacacaggggtacagaaggaataaattataaggatatgtttggagagttgccagtagtttagtattgctagagaagatggtgcaaggtaaaaaataatcaggaatgagactggatgtgtcagcagggtctacatcacaaagggccttaccttgatgtaaaattactttagagctgtctttttaagagagtattactgtcaatgtagattaaacacacaaacacacccagaccaagaggcaacaggatggagtataatctcctctattggattccttaggagccctgagggatttgttgaattactggtcccatgaacaaaatagctgtattgagagttggtcctttttatgtgacctaagaaatcatcaataaatatcaggcaatttctaaaagtaaagaatatatcatgaatattttcagggtttagggtaataataataatgttggcagaggtagtgatatcatcaaatgcctaagaaaatgaacctctcatatatatgatgactttcaggttaggattgatgaccaccttttaaaaaccgaggtaaaattcacacacagtgaaatgcacagatcttaagtgtaaaatctgtgagttttggtgtactccttaccctaatcaagacatcaaagccaggtgcagtggctcactcttgtaatcccaacactgtgggaggctgaggcggaaggatggcttgaggccaggagtttgagataagcctgggcatagcgagactctgtctctacaaaaaattagtcggacccagttgcacgctcctgtagtccagctactcagaaggctgaggcaggaagatcccttaagtccaggaatttgagggggcagtgagttatggtcatgccagtgcactccaacctaggtgacaaagtgagaccaccatctagaaaaataaagacttagaacatttccatcacctcagatagttcctttgttccctcttctccctatgttagtccccaccactcttttgatttgtcaccataggttggttttacctgttcttgaactttgtaaaaagggaatcatacaatatggacttttctgtttctggcttttttcactcaacatattgtttttgagatccattcatattgttgtatgtaccagtggtgtgttcctttttattgctaagtagattttcattatatgaatatacctcaatctgttgattcttctatttgatagacatttgggttgttttctatgaacattcttatacaaagcttcttgtagacatacgttttttatttttcttgggtaaatacctaggagtagaattgctgggtcataggttaaatatatgtttaacttaataactaataatttctaaaaaatggcactattatttattaggctggtgcaaaagtaattgcaattcttgccttttttttttttttttgagatggaatcttgctctgtcgccaggctggagtgcaatggcacgatctcagctcactgcaacttctgcttcccgggttcaagcgattcccttgcctcagcctcccaagtagctgggactacgggcgtatgccaccatgcctggctaactttttgtattttagtagagacggggtttcaccatgttggccaggatggtcttgatctcctgatctcgtgggttcttgcaatttaaatttaaatttaaaataattgcaagaactgtagttacttttgcaccagcctgatatatttttgccagcaatgcatgcaagttttagctgttccacatccttgccagcatttagtgttgtcattttaattttatttattctaacaggtgtgaaatggcacctcattatgattttaatatgcgtttccctgatgactaagactttgagtgacttttcctgtgcttattggccattcttatatcttcttttgtgaagtgttcatttcattctgttgtccatttttaattggctgtcttgattactgatttataagacttctttatagtcagataggtgctttgcaaatatttcctcccagtctatggactgtttttttttttaaattttcctaatgggagctttgttaagattaacaggtatttctcatttttttaaagtctaatttattcctttttaattttatgattaattctttttatgtcttctctagtgaatatttgtctaccccaagtttgcaggagatagtcctacatttttggtcataagtgttatgatttcagctttttatatttactgtgatcctctttaaattaatttttgtgtgtatgaagtaggggatgagattgattttttttctatgcttattcagtgttccatagcacctttgtttaaaaaactttgttttctgcactgatttgccttcattcctttgtcaaaaattaatttgctatatatgtgtaggattattatgatacactttcttctgttccattgatctttgtttttttttttatctccatgaccacaccatattgtcttaattatgtaacttaatagaaactcttgaaatctggcatatttcaagattggtttgtcccttctagattctttgcttttctctataaattacagtatcatcttactaatttctgtcaaaaaaaaatcctttgggaccgtgattaggagtctcaagctatttacccatttgggaaaagctgacaacttaacaatatagaatcttccagtccatgtacatgtattttttacttactagttcttctttattgtagcagtgttttgtgacttcagggttgagtcttgcacatcttttgtttatttcatccctaagtattttatgttttttgatgctgtcattaaagtattatttaaaaatttcataactcaattgtttggggctaatacaggaataccttggagatactgaggatttggttccaggctaccccaataaagcaaatattgcaataaagcagtaaatcaaatatttcaataaagcgagtcacatgaactttttggtttcccagtgcgtataaaagttatgtttacactatgctgtagtctattaagtgtgaaatagcattatgtctaaaaacaatgtacataccttaattaaaacatacttcattgctaaaaaatgctaacgatcatctgaaccttcagcaagccatatctttttgctggtggagggtcttgcctccatgtcagtggctgctgactgatcagattggtggttactgcaggttggagtgattgtgacaatttcttaaaataagacaacatgaagtttgacatatcagttgactcttctttcgtgaatgattctctgtatcatgcgatgctgtttgatagcattttacccagagtagaacttctttcaaaattggagtccgtccttcccctcaaaccctgccactgttttatcaactaagtttatgatggcgtaaattttttgttgtcatttcaacagtgtttatagcttcttcaccaggagtagattctatctcaaaaaaccacttttcttgattaactgtaaaaagtaactcctcatccagtcaagtttgatcatgagatcacagcaattcagtcacatcttcaggctcctcttataattctaattctcttgctattaaaaccatgtctgcagttacttcctccactgaagtctcgaacccctcaaagtcatccatgagggttggaatcagcttcttccaaagtcctgttaatgttgatatttttatctactcccatgaatcatgaatgttctgaatggcattcagaatggcgaatcctttccagaatgttttccattgattttgcccagatccaccagaagaatcaattatctgtggcaactctaggcttatgaaatgtatttcttaaataataaaacttgaaagttgaaatgcctccttcatccatgggctacagaatggataatgtgttagcagacatgaaaacattaatctccttgtacatgcccatcagcactcttgggtgaccaggttcattgccaatgaacaataatattttgaaagaaatcttttttctaagcagtaggtctcaagagtcctaaaatattcataaaaccatgctgtaaacagatacattgtcatccaggctttgatgtaccatttatagaacacaggcagagtagatttagcataattcttaagggccccagggtcttcagagttgcaaatgagcattagcttcaacttaaaatcagcagctgcattaacccctagtaaaagagtcagtctgtcctttgaagctttgaagccaggcattgacttctcctctctagctatgaaagtcctaggtggcatcttatttcaacagaaggctgtttcatctatgttgaaaatctgttgtttagtataaccaccttcattgatgatcttagctagagctactgagtaacttattgcagcttctccattagcacttcctgcttcaccttgcatgtctatgttctagaggtggcttctttccttaaacctcataaaccaatctctgctgtcttccaacttttcttctgcagcttccttacctctctcagccttcaaagatttgaagagagttaggtccttactctggattaggctttgggttaagagaaggttgtggctggtttgatcttgcatccagaccagtaaaactctttccgcatcagcaataaggctgttttactttcttatcattcgtgtgttcactggagtagcacttttaatttccttcaagaacttttcctttgcattcacaacttggctgactgtttcatgcaagaggcctagtttttggcttatcttggcttttcacctgccttcctcagtaagctttgtcatttctagcttttgatttaaagtgagagacataggactattcttttcacttgaacacctagaggttactgtaaggcttggcctaatttcattattgttgtgtctcagggaatatggaggcccaaggagaggaagagagactgggaatggccagtcagtagagcagtcagaacacaaaacatttatcgataaagttcactatcttatacgggtacagctcgtggtgctccaaaataactataatagcaacaccaaagatctctgatcacaggtcaccataacagatataataataatgataaagtttgagatattgtgagaattaccaaaatgtgacagagagacatgaagtgaccacatgctggtggaaaaatggctccagtagacttgattgactcagggttgccacaaaccttcaatttgtttttttttttttgtttcttttttttaaatatctatgaagcacaataaagcaaaatgcagtaaagtgaggtatgcctacatacagaaatataattgatttttttgtgtatattgaccttatatcccatgaccttgctaaattaacttattaattttagtatttgttttgtagatttctgatcattttccacatgacaattatgtcatctgcaaataaagacagtttgaccatttggtatggaaatgtataagaaatacatgagctatatttaaagataaagaggccaggtgcagtggcatagggctgtaatcctagtactttgagaggccgaggcgggcacattgcttgaacacaggagtttgagaccagtctgggcaacatggtgaaacctcgtctctgcaaaaaatacaaaaattagccaggcatggtggtgcatgcctgtagtcccagctatttgggggcctgaggtaggagaatcacttgagcccgggaggtcaaggctgcagtgagccgtgattgcaccactgtactccagcctgggcgacagagtgacatcctgtctcaaaataaaattaaattaaagataaggagaccaagaagcaaaaacacaaagaaaaccataatttaaaaataaaaatagctcttaattttttcattgttccttgaaagagaccttactgagaatgtagttggctagtttgaacctggccgagtatttaattcctcctattactttgtcttttctatcatcttttggttttttcttatatctttgagagtgctgtctgctttgattcaaccttctcaacttggattactgtgaattctttttttcctttatggatacctagaagtaattgtcacctttgagagtggtgatactctctcggtgactttagcaatccatttaatttcactgggcgtcagtttccacatctataaatgataggattttactatattactttctcagggctcttcccaactcctttgagttcttcatcttacctcacagtacatcagaggcaggcaggcaagtacaaaaagggtgttttagagctttactgttataggatttattgtattccctgttatttcattgcgaatatgctagataattaaaaatcaattgagttcattattaattctatgtatcctccaggtttgtcatttatatctgaggctctttgttgagtttactgttaactcatttacatttttctataacttata

SEQ ID NO: 2 - DNA, 53 bp, Artificial Sequence
  Chemically synthesized primer
atttggagtgaccaaaggtttcaagagaacctttggtcactccaaattttttg

SEQ ID NO: 3 - DNA, 57 bp, Artificial sequence
  Chemically synthesized primer
aattcaaaaaatttggagtgaccaaaggttctcttgaaacctttggtcactccaaat

SEQ ID NO: 4 - DNA, 57 bp, Artificial sequence
  Chemically synthesized primer
cgcaagctgaccctgagttcattcaagagatgaacttcagggtcagcttgctttttg

SEQ ID NO: 5 - DNA, 66 bp, Artificial sequence
  Chemically synthesized primer
aattcaaaaagcaagctgaccctgaagttcatctcttgaatgaacttcagggtcagcttgcgggcc

SEQ ID NO: 6 - DNA, 26 bp, Artificial sequence
  Chemically synthesized primer
tcgggccgcggcaagactggcggcaa

SEQ ID NO: 7 - DNA, 27 bp, Artificial sequence
  Chemically synthesized primer
gtactcctgggaggcctgggtggcctt

SEQ ID NO: 8 - DNA, 23 bp, Artificial sequence
  Chemically synthesized primer
actctgcgttgataccactgctt

SEQ ID NO: 9 - DNA, 23 bp, Artificial sequence
  Chemically synthesized primer
aagcagtggtatcaacgcagagt

SEQ ID NO: 10 - DNA, 26 bp, Artificial sequence
  Chemically synthesized primer
aagcagtggtatcaacgcagagtggg

(19) United States
(12) Patent Application Publication
(10) Pub. No.: US 2009/0047672 A1
(43) Pub. Date: Feb. 19, 2009

(54) Telomerase RNA Subunit and Methods of Use Thereof

(76) Inventors: William C. Hahn, Newton, MA (US); Richard Possemato, Brighton, MA (US); Kenkichi Masutomi, Brookline, MA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/883,965

(22) PCT Filed: Feb. 7, 2006
(86) PCT No.: PCT/US06/04188

§ 371 Date: May 20, 2008

Related U.S. Application Data
(60) Provisional application No. 60/650,614, filed on Feb. 7, 2005.

(51) Int. Cl. C12Q 1/68; C12N 15/11; C12N 15/00; C12N 5/06; C12N 9/12

(52) U.S. Cl. 435/6; 536/23.2; 435/320.1; 435/325; 435/194; 435/375

(57) ABSTRACT

The present invention provides a novel telomere associated RNA (hTERC-2) that mediates the DNA repair function of telomerase.

### STATEMENT OF GOVERNMENT SUPPORT

This invention was made with U.S. Government support under National Institutes of Health/National Cancer Institutes grant CA94223. The government has certain rights in the invention.

### FIELD OF THE INVENTION

The invention relates to generally to compositions and methods modulating cell senescence.

### BACKGROUND OF THE INVENTION

Telomerase is a specialized ribonucleoprotein (RNP) reverse transcriptase that is essential for telomere maintenance. Telomerase uses an internal RNA template to synthesize telomeric repeat sequences onto chromosome ends. Deletion of the essential RNA component of telomerase leads to progressive telomere shortening, chromosome instability and cell death

Both telomere length and telomerase activity have been implicated in cellular senescence and cancer. In most somatic cells, telomerase activity is not detected and telomeres shorten with each division. Artificial elongation of telomeres by ectopic hTERT expression in primary human cells leads to telomere elongation and a bypass of cellular senescence, suggesting that telomere shortening may trigger cellular senescence in primary human cells. During immortalization of mammalian cells in culture, telomerase is activated, telomere length is stabilized, and cells continue to proliferate, suggesting that telomerase activation and telomere stabilization are required for the long term growth of cancer cells. Telomerase activity is present in the vast majority of human tumors while little activity is found in the normal tissues from which the tumors were derived.

### SUMMARY OF THE INVENTION

The invention is based upon the discovery of a novel function telomerase RNA subunit referred to herein as TERC-2. The cloned TERC-2 fragment is contained within the Human UVRAG intron 6 sequence (SEQ ID NO:1). Accordingly, in one aspect the invention provides telomerase RNA subunit containing at least 100 nucleotides of SEQ ID NO:1. The telomerase RNA subunit is at least 250, 500, 1000 or more nucleotides in length. The telomerase RNA subunit binds a telomerase catalytic subunit (TERT) polypeptide. Also included in the invention are vectors containing the telomerase RNA subunit and cells containing the vector.

In another aspect, the invention provides a complex contain a telomerase catalytic subunit (TERT) polypeptide and TERC-2.

Cell senescence is induced by contacting a cell with a compound that inhibits the interaction between a telomerase catalytic subunit (TERT) polypeptide and TERC-2 or decreases the activity or expression of TERC-2. The compound is an anti-TERC2 antibody, a TERC-2 anti-sense nucleic acid or a TERC-2 RNAi. The cell is a cancer cell. The cell is contacted in vivo, in vitro or ex vivo. Optionally, the cell is further contacted with a cytotoxic agent such as a chemotherapeutic compound. Senescent cells are no longer capable of dividing yet remain metabolically active. Cell divison is measured by using methods know in the art to detect DNA synthesis. For example cell division is determined by the BrdU incorporation assay.

Cell viability is enhanced by contacting a cell with a composition containing TERC-2. By viability is meant that the cell is excludes a vital dye, such as trypan. Viable cells are also capable of proliferation, differentiation, growth and development. Viability is measured by methods known in the art such as trypan blue staining. The cell is contacted in vivo, in vitro or ex vivo.

The invention further includes a method identifying inhibitors or enhancers of the telomerase-TERC-2 subunit interaction bringing into contact a telomerase protein, a TERC-2 RNA and a test compound under conditions where the telomerase protein and the TERC-2 RNA, in the absence of compound, are capable of forming a complex; and determining the amount of complex formation. A decrease in the amount of complex formation in the presence of the test compound compared to the absence of the test compound indicates that the test compound in an inhibitor of the telomerase-TERC-2 subunit interaction. Similarly, an increase in the amount of complex formation in the presence of the test compound compared to the absence of the test compound indicates that the test compound in an enhancer of the telomerase-TERC-2 subunit interaction.

Compounds that bind TERC-2 are identified by contacting TERC-2 with a test agent and determining whether the test agent binds to TERC-2. In some aspects, the TERC-2 contains a label such as a fluorescent label, or a radioactive label.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is a series of photographs showing the effects of hTERT suppression on H2AX phosphorylation. BJ fibroblasts expressing either a control shRNA or an hTERT-specific shRNA were irradiated (10 Gy), incubated for 1 h, fixed and stained with a rabbit anti-H2AX Ab.

FIGS. 1B and C are photographs of an immunoblot of DNA damage proteins. BJ cells stably expressing either a control vector, an hTERT-specific shRNA, a 3′UTR hTERT-specific shRNA or a 3′ UTR hTERT-specific shRNA together with WT hTERT were irradiated (10 Gy), incubated for the indicated time, and lysed. Whole cell lysates (100 μg) were resolved by SDS-PAGE and immunoblotted with the indicated antibodies. pBRCA1=phosphorylated BRCA1.

FIG. 1D is a photograph of a Western blot showing BJ cells stably expressing either a control vector, an hTERT-specific shRNA, a 3′UTR hTERT-specific shRNA or a 3′ UTR hTERT-specific shRNA together with WT hTERT were treated with indicated chemotherapeutic drugs at 10 μM for 4 hrs and lysed. Whole cell lysates (100 μg) were resolved by SDS-PAGE and immunoblotted with the indicated antibodies.

FIG. 1E is a photograph of a Western Blot showing DNA damage response in WI38 fibroblasts. WI38 cells expressing the indicated shRNA vectors were irradiated as in (B) and immunoblotting on whole cell lysates (100 μg) was performed.

FIG. 2A is a series of photographs showing the effects of ionizing radiation on telomere length and co-localization with H2AX. BJ fibroblasts expressing either a control shRNA or an hTERT-specific shRNA were exposed to ionizing radiation (10 Gy) and incubated for 1 h. Fixed cells were hybridized with a fluorescein isothiocyanate (FITC)-conjugated telomere-specific PNA probe. After fluorescence in situ hybridization (FISH), cells were further stained with a rabbit anti—H2AX Ab (red) and DAPI (blue). Panels are shown at 1000× magnification.

FIG. 2B is a photograph of a showing the effects of irradiation on telomere length. BJ fibroblasts expressing either a control shRNA or an hTERT-specific shRNA were irradiated (5 Gy); genomic DNA was isolated immediately (0 h) or 6 h later; and telomere length was determined by Southern blotting for telomere restriction fragments (TRF).

FIG. 2C are bar charts showing quantitative-FISH (Q-FISH) telomere length analysis in BJ fibroblasts expressing a control shRNA or an hTERT-specific shRNA. For each cell line at least 400 chromosomes were analyzed, and the mean fluorescence intensity correlated to telomere length is shown. Since the cells used in this study arrest after irradiation, Q-FISH could not be performed on irradiated cells. No significant difference in the number of telomere ends lacking a fluorescence signal was observed in either of these cell populations.

FIG. 2D is a photograph showing the effects of irradiation on telomeric single-stranded overhangs. BJ fibroblasts expressing either a control shRNA or an hTERT-specific shRNA were irradiated (5 Gy); genomic DNA was isolated immediately (0 h) or at the indicated time points; and the telomeric 3′-overhang ligation assay (T-OLA) was performed. Molecular weight markers are noted in nucleotides to left of panel. PCR for GAPDH confirmed that equivalent amounts of DNA were analyzed in each lane.

FIG. 2 E is a schematic summary of hTERT mutants. X represents substitution of aspartic acid and valine residues at position 731 and 732 with alanine and isoleucine. The black bars represent sites where the endogenous hTERT sequence was substituted with the peptide sequence NAAIRS.

FIG. 2F is a line graph showing the effects of hTERT mutant expression on the replicative lifespan in fibroblasts that lack endogenous hTERT expression by suppression with a 3′UTR hTERT specific shRNA. BJ cells expressing a control shRNA (closed circles), a 3′UTR hTERT-specific shRNA with vector control (closed triangles), a 3′UTR hTERT-specific shRNA with wild-type (WT) hTERT (closed squares), a 3′UTR hTERT-specific shRNA with DN hTERT (open diamonds), a 3′UTR hTERT-specific shRNA with N-DAT92 (open squares), a 3′UTR hTERT specific shRNA with N-DAT122 (open circles) and a 3′UTR hTERT-specific shRNA with CDAT1127 (open triangles) are shown. Error bars represent mean±SD for three independent determinations. In some cases, the symbol covers the error bars.

FIG. 2G is a photograph of a blot showing the effects of hTERT mutant expression on the DNA damage response in fibroblasts that lack endogenous hTERT expression. BJ cells expressing a 3′UTR hTERT-specific shRNA together with a control vector (Vector), WT hTERT, DN hTERT, N-DAT92, N-DAT122 and C-DAT1127 were irradiated (10 Gy), incubated for 1 h, and lysed. Whole cell lysates (100 μg) were resolved by SDS-PAGE and immunoblotted with the indicated antibodies. Telomerase activity was measured using the TRAP assay. HT refers to heat-treated samples. IC refers to the internal PCR control for the TRAP assay.

FIG. 3A is a photograph of a Western blot showing suppressing hTERT expression alters H2AX accessibility. Extraction of H2AX from chromatin. BJ cells expressing either a control vector, an hTERT specific shRNA, a 3′UTR hTERT-specific shRNA or a 3′ UTR hTERT-specific shRNA together with WT hTERT were irradiated (10 Gy), incubated for the indicated time, and lysed with RIPA buffer. Whole cell lysates (100 μg) were resolved by SDS-PAGE and immunoblotted with the indicated antibodies.

FIG. 3B is a photograph of a Northern blot showing suppressing hTERT expression alters H2AX accessibility. H2AX mRNA expression. Total RNA (500 ng) was used for RT-PCR with primers specific for H2AX and -actin.

FIG. 3C is a photograph demonstrating precipitation of H2AX from chromatin under acidic conditions. Acid precipitation of H2AX from BJ cells expressing either a control vector, an hTERT-specific shRNA, a 3′UTR hTERT-specific shRNA or a 3′ UTR hTERT-specific shRNA together with WT hTERT.

FIG. 3D is a photograph showing extraction of histones under low and high ionic strength. The indicated cells were lysed with low salt buffer and high salt buffer and immunoblotted with the indicated antibodies.

FIG. 3E is a photograph showing the extraction of core histones from chromatin. BJ cells expressing either a control vector, an hTERT-specific shRNA, a 3′UTR hTERT-specific shRNA or a 3′ UTR hTERT-specific shRNA together with WT hTERT were lysed in RIPA buffer. Whole cell lysates (100 μg) were resolved by SDS-PAGE and immunoblotted with the indicated antibodies.

FIG. 3F is a photograph of a Western blot showing the effects of hTERT suppression on chromatin alterations induced by trichostatin A (TSA). Cells were treated with TSA (10 μM) for 8 h. Phosphorylated ATM and total ATM protein levels were determined by immunoblotting.

FIG. 3G depicts micrococcal nuclease digestion of nuclei derived from cells expressing a control vector, an hTERT-specific shRNA, a 3′UTR hTERT-specific shRNA or a 3′ UTR hTERT-specific shRNA together with WT hTERT. Nuclei isolated from 1×10⁶ cells were treated with micrococcal nuclease for the indicated time, subjected to agarose gel electrophoresis and stained with ethidium bromide.

FIG. 3H is a photograph of a western blot showing histone tail modifications. BJ cells expressing either a control vector, an hTERT-specific shRNA, a 3′UTR hTERT-specific shRNA or a 3′ UTR hTERT-specific shRNA together with WT hTERT were lysed in RIPA and immunoblotting with the indicated antibodies was performed.

FIG. 4A is a line graph depicting the effects of hTERT suppression on clonogenic growth after ionizing radiation (IR). BJ cells expressing a control vector (closed circles), an hTERT-specific shRNA (triangles), a 3′ UTR hTERT-specific shRNA (closed squares), a 3′UTR hTERT-specific shRNA together with wild-type (WT)-hTERT (diamonds), and WT-hTERT (open circles), respectively, were exposed to -irradiation. Relative cell survival was calculated as the percentage of viable cells after irradiation relative to cells not exposed to ionizing radiation. Mean±standard deviation are shown for each point. In some cases, the error bars are covered by the symbol.

FIG. 4B is a bar chart showing the effects of hTERT suppression on DNA repair. BJ cells expressing a control vector, an hTERT-specific shRNA, a 3′UTR hTERT-specific shRNA, a 3′ UTR hTERT-specific shRNA together with WT hTERT, or WT hTERT were irradiated (2 Gy). The fraction of DNA breaks induced by ionizing radiation that was repaired at 4 h was measured by pulse field gel electrophoresis and normalized to the control shRNA samples as described in Methods. Each bar represents the mean±SD, and the experiment shown is representative of 3 independent experiments.

FIG. 5A is a photograph showing the effects of hTERT-specific shRNAs on telomerase. BJ fibroblasts were infected with a GFP-specific shRNA (Control), an hTERT coding sequence-specific shRNA (hTERT shRNA) or an hTERT 3′untranslated region-specific shRNA (hTERT 3′ UTR shRNA)

FIG. 5B is a photograph showing the results of the IP-TRAP assay. After synchronization with serum starvation and aphidicolin treatment, IP-TRAP was performed as described. RNase refers to treatment with RNase prior to TRAP assay.

FIG. 6 is a photograph of a Western Blot showing Human BJ fibroblasts expressing a control vector or the DN hTERT mutant exposed to ionizing radiation (10 Gy). Immunoblotting with the indicated antibodies was performed similar to the panels shown in FIG. 1c. The signal of H2AX observed in cells expressing the DN hTERT mutant is decreased by 80% compared with cells expressing a control vector.

FIG. 7 is a schematic showing the nucleotide sequence of Human UVRAG intron 6 sequence. (SEQ ID NO:1)

### DETAILED DESCRIPTION OF THE INVENTION

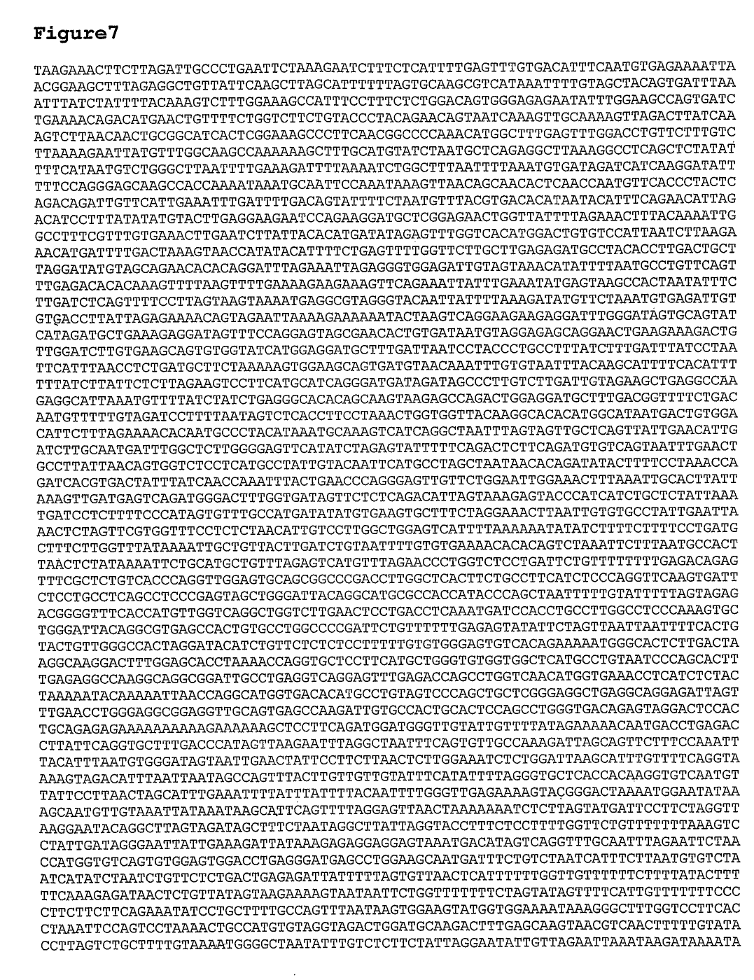

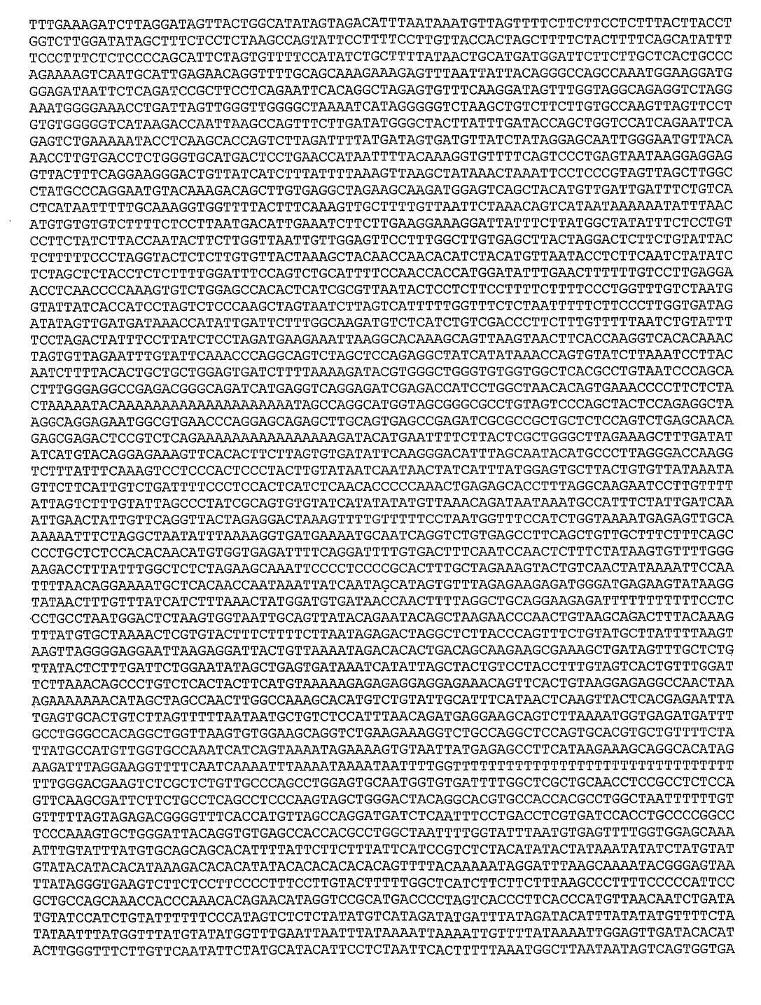

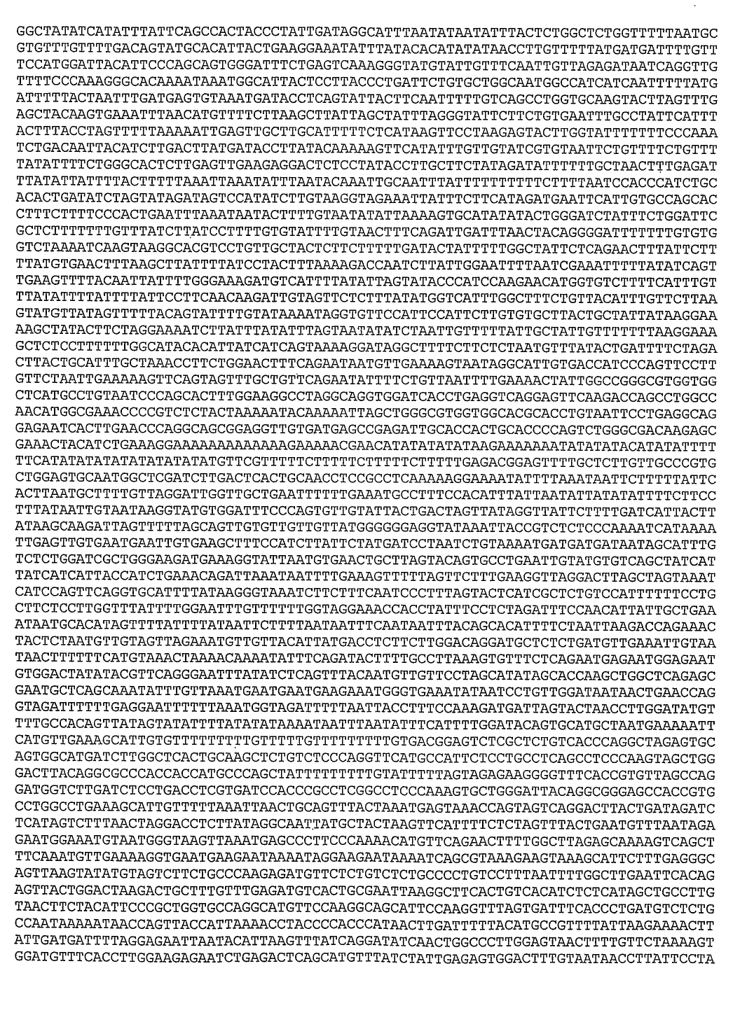

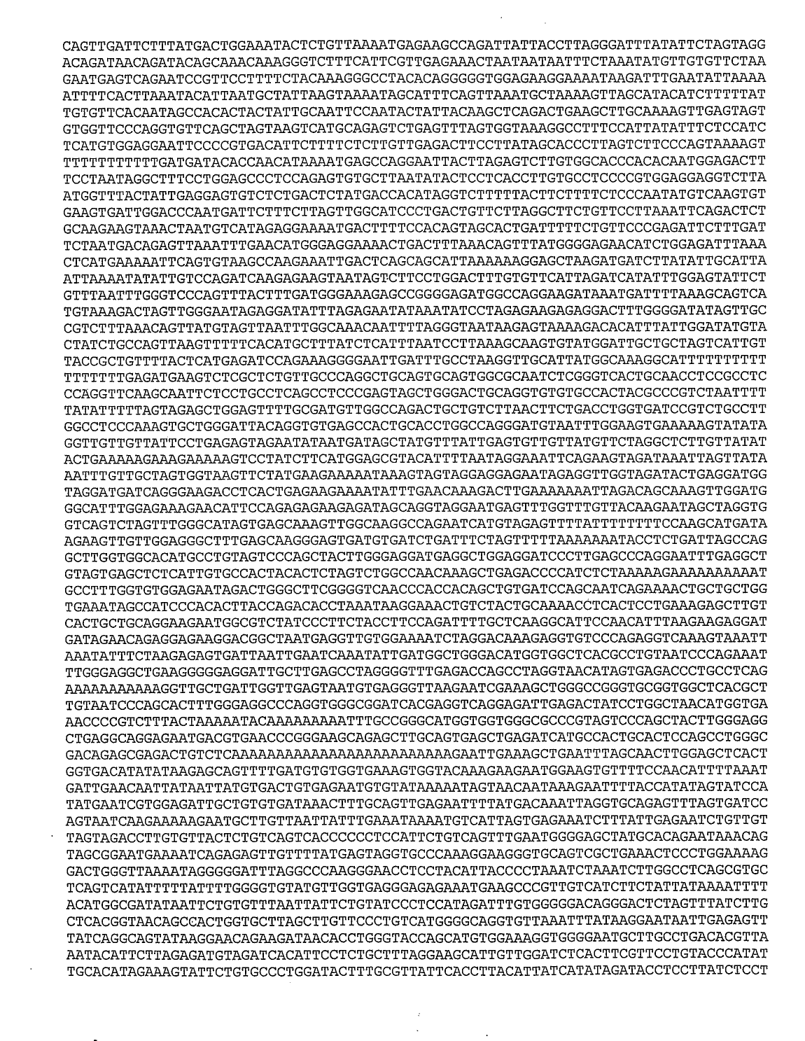

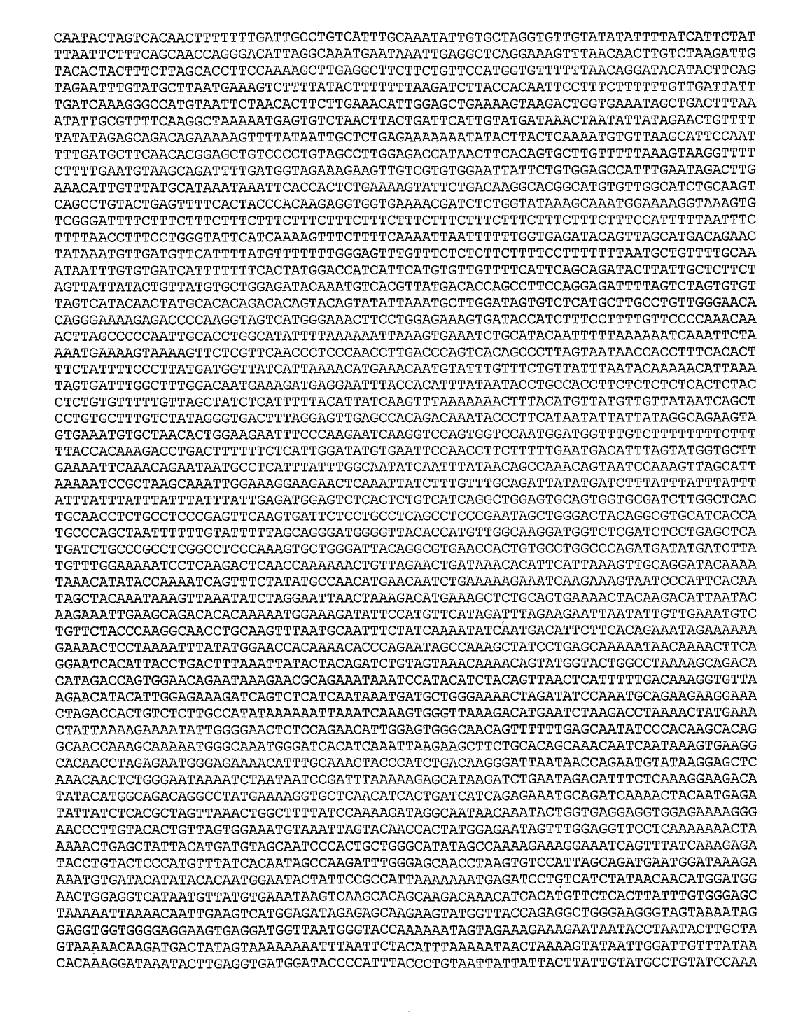

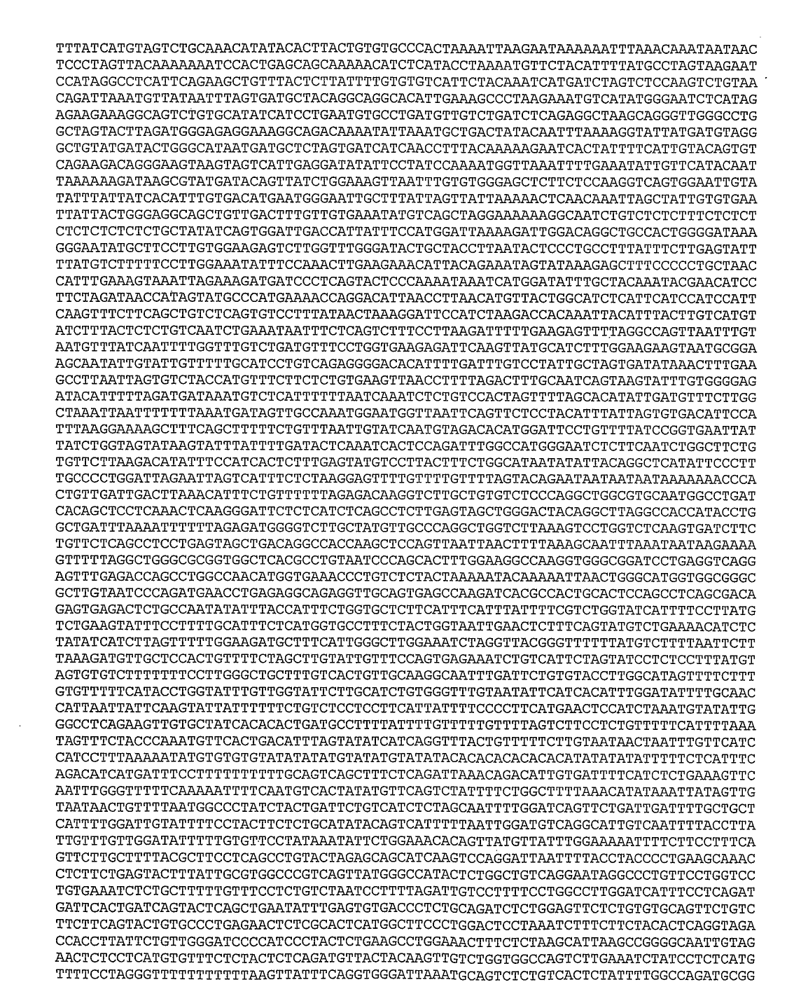

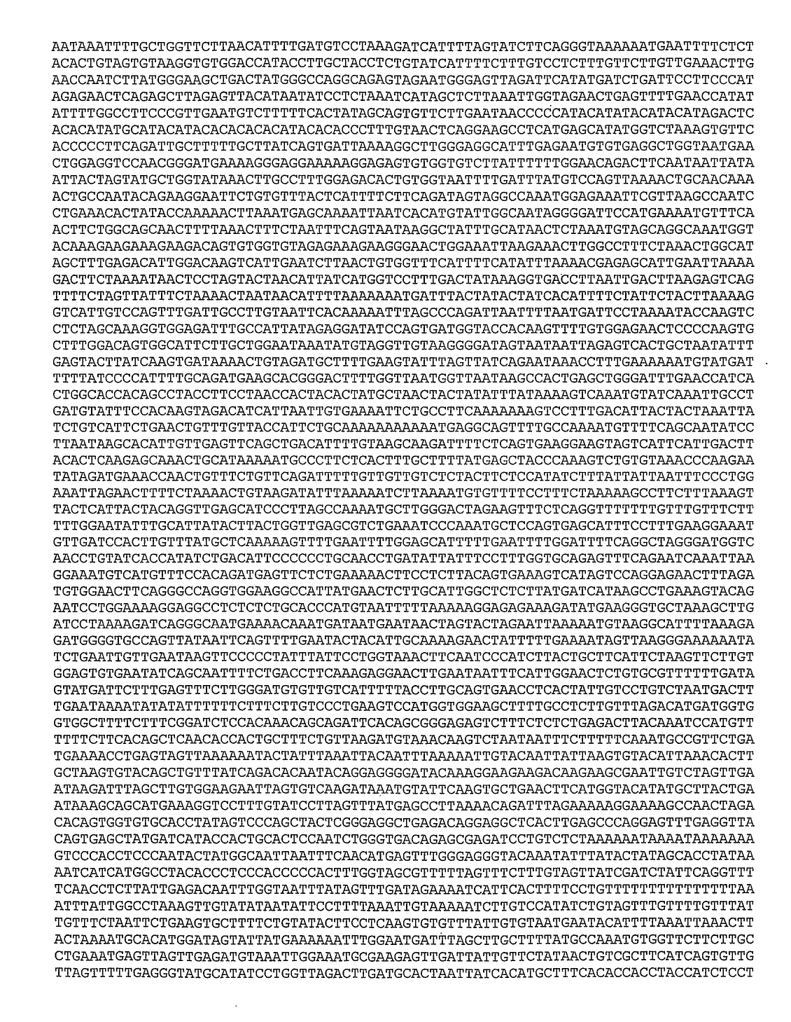

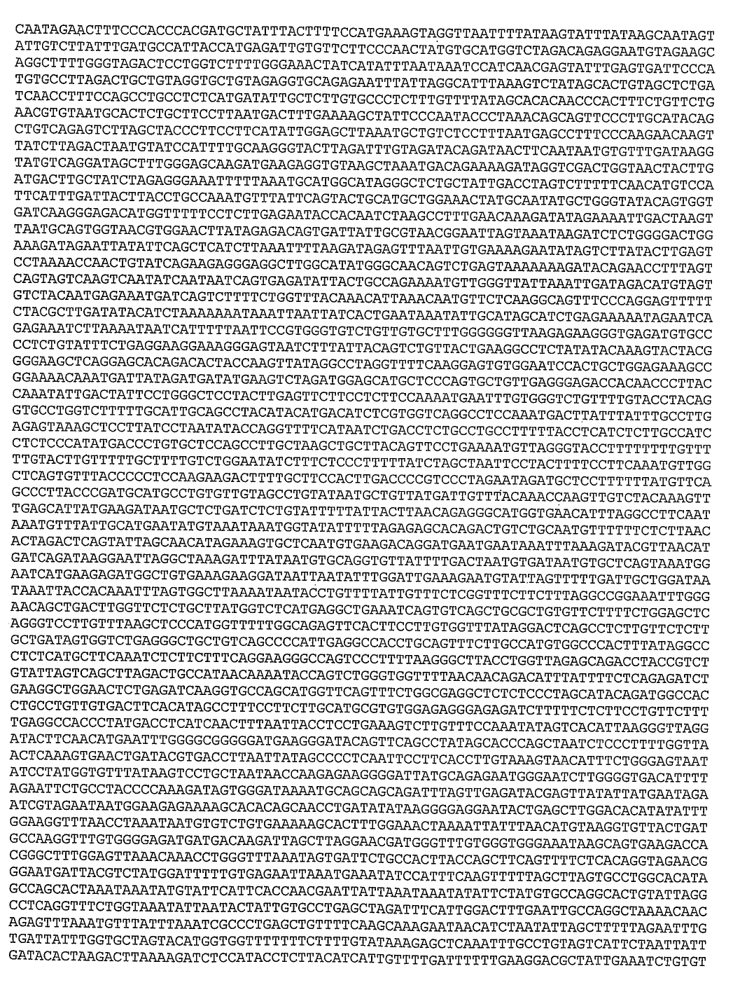

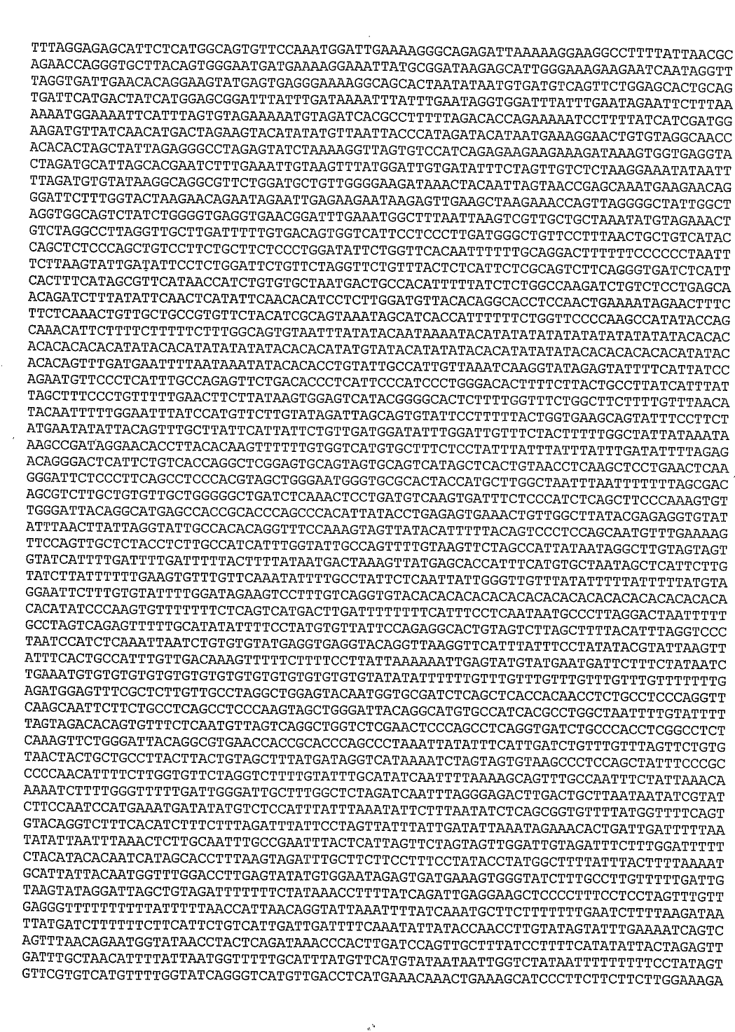

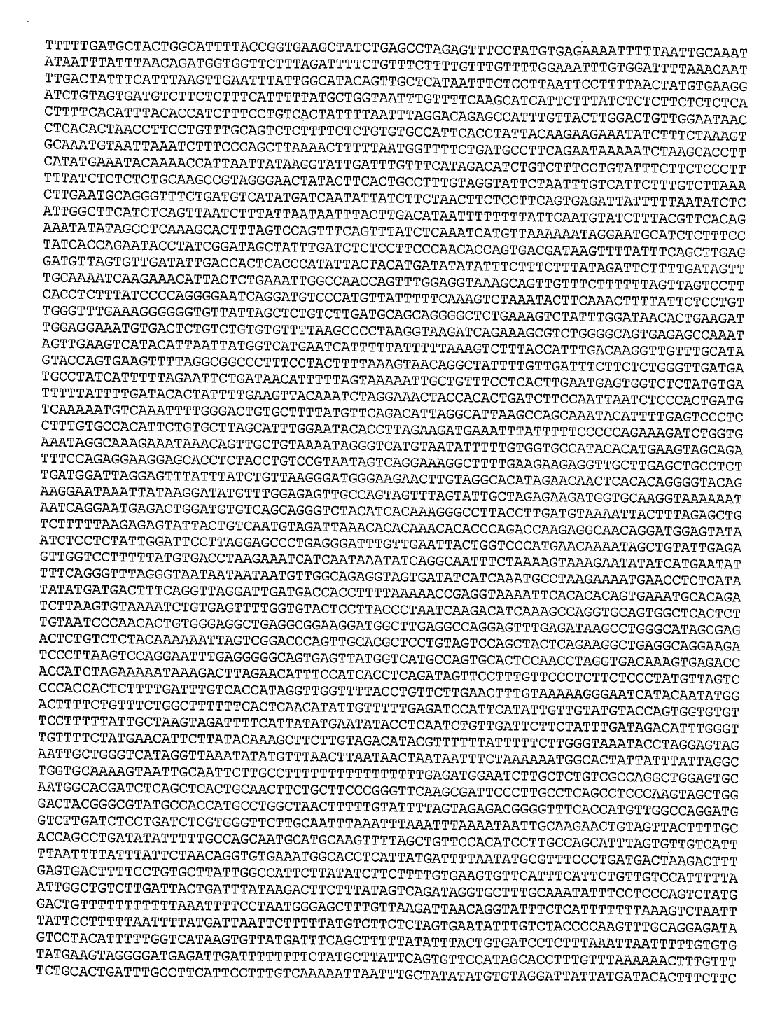

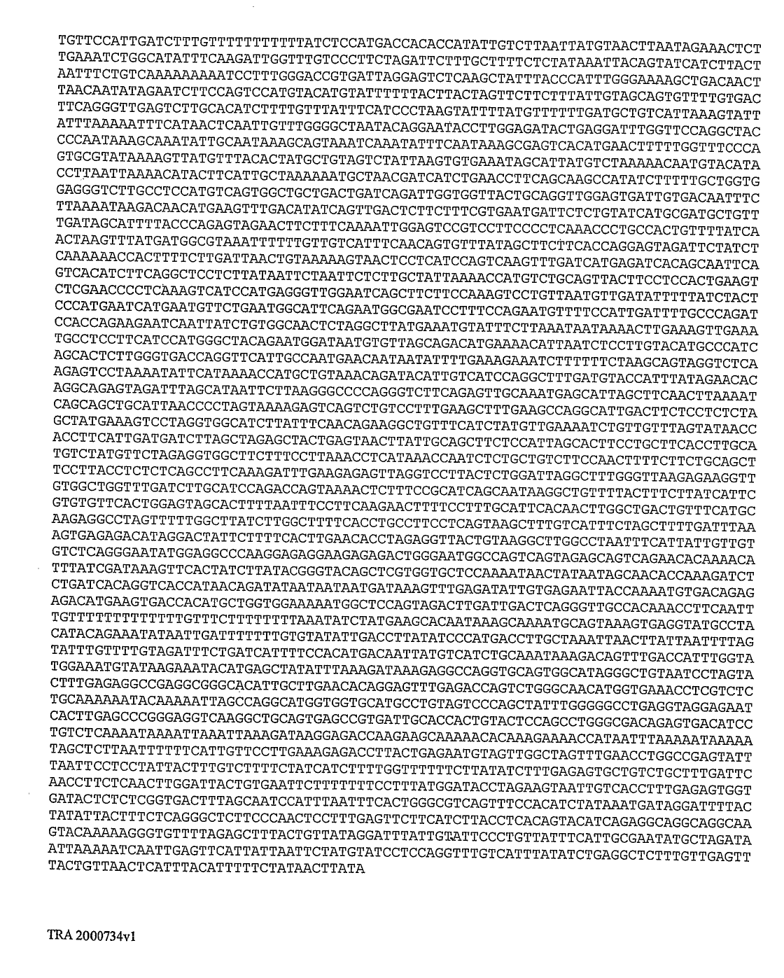

The invention is based upon the surprising observation that suppression of human telomerase catalytic subunit (hTERT) expression abrogates the DNA damage response to chemical or physical agents that induce DNA double strand breaks. More particularly, the invention is based upon the discovery of a novel function telomerase RNA subunit referred to herein as TERC-2. The cloned TERC-2 fragment is contained within the Human UVRAG intron 6 sequence (SEQ ID NO:1, FIG. 7). Telomerase is a ribonucleoprotein know to be responsible for the maintenance of telomeres, the physical ends of chromosomes. The telomerase enzyme is made up of an essential core as well as several accessory proteins. The core telomerase consists of the RNA component (Telomerase RNA, TR) and the catalytic subunit (Telomerase Reverse Transcriptase, TERT). The RNA component of the protein contains the template for replication of the DNA.

Constitutive expression of telomerase in human cells prevents the onset of senescence and crisis by maintaining telomere length homeostasis. Recent evidence suggests that telomerase is dynamically regulated in normal cells and contributes to malignant transformation independent of its ability to lengthen telomeres. Normal human somatic cells exhibit a limited replicative lifespan and eventually enter a growth arrest state termed replicative senescence triggered by dysfunctional telomeres. However, other stimuli such as oncogene activation, increased oxidative potential and genotoxic damage also trigger a cell cycle arrest state that shares both morphologic and functional similarities with replicative senescence.

Furthermore, recent work indicates that senescent human cells show evidence of activation of the DNA damage response pathway. Although overexpression of telomerase maintains telomere length and facilitates human cell immortalization, accumulating evidence also suggests that telomerase itself plays an additional role in protecting karyotypic stability by “capping” chromosomes. Indeed, constitutive overexpression of TERT facilitates malignant transformation independent of its effects on telomere length and renders cells more resistant to apoptosis. These observations connect telomerase expression, DNA damage responses and senescence, suggesting that hTERT may contribute to the cellular response to genotoxic insults The present invention shows that suppression of human telomerase catalytic subunit (hTERT) expression abrogates the DNA damage response to chemical or physical agents that induce DNA double strand breaks. Loss of hTERT does not alter short-term telomere integrity but instead affects the overall configuration of chromatin as assessed by sensitivity to micrococcal nuclease digestion, accessibility of the histone H2AX and post-translational modifications of the core histones H3 and H4. Human cells lacking hTERT exhibit increased radiosensitivity, show impaired capacity for DNA repair and accumulate fragmented chromosomes. These studies demonstrate a second function of hTERT which involved in the cellular response to DNA damage by regulating chromatin state. These results further indicate that blockade of this DNA repair function will be of therapeutic benefit in diseases mediated cell immortalization.

Accordingly in one aspect the present invention provides a novel telomerase RNA subunit, which participates in this second telomerase function involved in the response to DNA damage. This novel RNA, TERC-2 was identified by using a monoclonal antibody specific for hTERT. The invention also provides methods of modulating cell senescence by inhibiting the DNA repair function of telomerase.

TERC-2 Nucleic Acids

The invention also features isolated polynucleotides encoding TERC-2. As used herein, “isolated” refers to a sequence corresponding to part or all of the TERC-2, but free of sequences that normally flank one or both sides of the TERC-2 sequence. An isolated polynucleotide is for, for example, a recombinant RNA molecule, provided one of the nucleic acid sequences normally found immediately flanking that recombinant RNA molecule in a naturally-occurring molecule is removed or absent. Thus, isolated polynucleotides include, without limitation, a recombinant RNA that exists as a separate molecule (e.g., a cDNA or genomic DNA fragment produced by PCR or restriction endonuclease treatment) independent of other sequences as well as recombinant RNA that is incorporated into a vector, an autonomously replicating plasmid, a virus (e.g., a retrovirus, adenovirus, or herpes virus), or into the genomic RNA of a prokaryote or eukaryote. In addition, an isolated polynucleotide can include a recombinant RNA molecule that is part of a hybrid or fusion polynucleotide.

“Polynucleotides” are at least about 14 nucleotides in length. For example, the polynucleotide can be about 14 to 20, 20-50, 50-100, or greater than 150 nucleotides in length. Polynucleotides can be linear or circular, and in sense or antisense orientation.

The polynucleotides of the invention have at least 70% sequence identity to the nucleotide sequence of SEQ ID NO:1. The nucleic acid sequence can have, for example, at least 80%, 90%, or 95% sequence identity to SEQ ID NO:1. Generally, percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Nucleic acid sequences can be aligned by visual inspection, or by using sequence alignment software. For example, MEGALIGN™. (DNASTAR, Madison, Wis., 1997) sequence alignment software, using default parameters for the Clustal algorithm, can be used to align polynucleotides. In this method, sequences are grouped into clusters by examining the distance between all pairs. Clusters are aligned as pairs, then as groups.

The invention also features polynucleotides that are at least 150 nucleotides in length and that hybridize under stringent conditions to the polynucleotide of SEQ ID NO:1 or to the complement thereof. Hybridization typically involves Southern analysis. See, for example, sections 9.37-9.52 of Sambrook et al., 1989, “Molecular Cloning, A Laboratory Manual”, second edition, Cold Spring Harbor Press, Plainview; N.Y. Stringent conditions can include the use of low ionic strength and high temperature for washing, for example, 0.015 M NaCl/0.0015 M sodium citrate (0.1.times.SSC), 0.1% sodium dodecyl sulfate (SDS) at 60° C. Alternatively, denaturing agents such as formamide can be employed during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is the use of 50% formamide, 5.times.SSC (0.75 M NaCl, 0.075 M sodium citrate), 50 mM sodium phosphate (pH 6.8), 0.1% sodium pyrophosphate, 5 times. Denhardt's solution, sonicated salmon sperm DNA (50 .mu.g/ml), 0.1% SDS, and 10% dextran sulfate at 42° C., with washes at 42° C. in 0.2×.SSC and 0.1% SDS.

RNA molecules containing one of the disclosed sequences are produced recombinantly using known techniques, by in vitro transcription, and by direct synthesis. For recombinant and in vitro transcription, DNA encoding RNA molecules is obtained from known clones, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al.

Detection of interactions between RNA binding proteins and RNA molecules can be facilitated by attaching a detectable label to the RNA molecule. Generally, labels known to be useful for nucleic acids can be used to label RNA molecules. Examples of suitable labels include radioactive isotopes such ³³P, ³²P, and ³⁵S, fluorescent labels such as fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, and biotin.

Labeled nucleotides are the preferred form of label since they can be directly incorporated into the RNA molecules during synthesis. Examples of detection labels that can be incorporated into amplified RNA include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res. 22:3226-3232 (1994)). A preferred nucleotide analog label for RNA molecules is Biotin-14-cytidine-5′-triphosphate. Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Method of Inducing Cell Senescence

Cell senescence is induced by contacting a cell with a compound that inhibits the interaction between a telomerase catalytic subunit (TERT) polypeptide and TERC-2. By inhibiting the interaction is meant that the compound inhibits the binding of TERT to TERC-2 or inhibits the activity of a TERC-2/TERT complex. The compound is a small molecule, polypeptide or nucleic acid molecule. Examples of small molecules include, but are not limited to, peptides, peptidomimetics (e.g., peptoids), amino acids, amino acid analogs, polynucleotides, polynucleotide analogs, nucleotides, nucleotide analogs, organic and inorganic compounds (including heterorganic and organomettallic compounds) having a molecular weight less than about 5,000 grams per mole, organic or inorganic compounds having a molecular weight less than about 2,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. For example the compound is a TERT or TERC-2 mimetic, an anti-TERT antibody or an anti-TERC-2 antibody.

Alternatively, cell senescence is induced by contacting a cell with a compound that decreases the expression or activity of TERC-2. A decrease in TERC-2 expression or activity is defined by a reduction of a biological function of the TERC-2. A TERC-2 biological function includes DNA repair. TERC-2 expression is measured by detecting a TERC-2 transcript. TERC-2 inhibitors are known in the art or are identified using methods described herein. For example, a TERC-2 inhibitor is identified by detecting a decrease in the DNA damage response to chemical or physical agents that induce double strand breaks. DNA damage is detected by methods known in the art such as the accumulation of fragmented chromosomes. For example, an increase of fragmented chromosomes in the presence of the compound compared to the absence of the compound indicates a decrease in TERC-2 activity. An TERC-2 inhibitor is also identified by detecting the inhibition of the interaction between TERC-2 and TERT.

The TERC-2 inhibitor is for example an antisense TERC-2 nucleic acid, a TERC-2-specific short-interfering RNA, or a TERC-2-specific ribozyme. By the term “siRNA” is meant a double stranded RNA molecule which prevents translation of a target mRNA. Standard techniques of introducing siRNA into a cell are used, including those in which DNA is a template from which an siRNA RNA is transcribed. The siRNA includes a sense TERC-2 nucleic acid sequence, an anti-sense TERC-2 nucleic acid sequence or both. Optionally, the siRNA is constructed such that a single transcript has both the sense and complementary antisense sequences from the target gene, e.g., a hairpin. Binding of the siRNA to an TERC-2 transcript in the target cell results in a reduction in TERC-2 in the cell. The length of the oligonucleotide is at least 10 nucleotides and may be as long as the naturally-occurring TERC-2 transcript. Preferably, the oligonucleotide is 19-25 nucleotides in length. Most preferably, the oligonucleotide is less than 75, 50, 25 nucleotides in length.

Cell senescence is characterized by a the inability of a cell to divide. The cell is any cell that expresses TERC-2, for example the cell is a cancer cell. Cells are directly contacted with an inhibitor. Alternatively, the inhibitor is administered systemically. Optionally, the cell is further contacted with a cytotoxic agent such as a chemotherapeutic compound.

The methods are useful to alleviate the symptoms of a variety of cell proliferative disorders. Cell proliferative disorders include cancer and cardiovascular diseases. Cancer is for example lymphoma, leukemia, myeloma, lung cancer, colon cancer, stomach cancer, brain cancer or pancreatic cancer. Efficaciousness of treatment is determined in association with any known method for diagnosing or treating the particular cell proliferative disorder. Alleviation of one or more symptoms of the cell proloferative disorder indicates that the compound confers a clinical benefit.

### Methods of Enhancing Cell Viability

Cell viability is enhanced by contacting a cell with a composition containing a compound that increases the expression or activity of TERC-2. The compound is for example, e.g., (i) TERC-2, e.g., SEQ ID NO:1; (ii) a nucleic acid encoding a TERC-2; (iii) a nucleic acid or polypeptide that increases expression of a nucleic acid that encodes a TERC-2 and, and derivatives, fragments, analogs and homologs thereof.

The nucleic acid compositions are formulated in a vector. Vectors include for example, an adeno-associated virus vector, a lentivirus vector and a retrovirus vector. Preferably the vector is an adeno-associated virus vector. Preferably the nucleic acid is operatively linked to a promoter such as a human cytomegalovirus immediate early promoter. An expression control element such as a bovine growth hormone polyadenylation signal is operably-linked to coding region the cell protective polypeptide. In preferred embodiments, the nucleic acid of the is flanked by the adeno-associated viral inverted terminal repeats encoding the required replication and packaging signal

The methods are useful to alleviate the symptoms of a variety of disorders characterized by aberrant cell death. Conditions characterized by aberrant cell death include cardiac disorders (acute or chronic) such as stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia, or myocardial hypertrophy or neurological disorders such as Amyotrophic Lateral Sclerosis, Alzheimer's disease, Huntington's disease and Parkinson's disease

### Methods of Screening for TERC-2 Modulating Compounds

The invention further provides a method of screening for compound that modulate TERC-2, e.g., inhibitors or enhancers.

In various methods, an inhibitor or enhancer of the telomerase/TERC-2 interaction is identified by contacting a telomerase protein, a TERC-2 RNA and a test compound under conditions where telomerase and TERC-2 are capable of forming a complex and the amount of complex formation is determined. A decrease in the amount of complex formation in the presence of the test compound compared to the absence of the test compound indicates that the test compound in as inhibitor of the telomerase-TERC-2 subunit interaction. In contrast, an increase in the amount of complex formation in the presence of the test compound compared to the absence of the test compound indicates that the test compound in as enhancer of the telomerase-TERC-2 subunit interaction.

The invention also provide a method of identifying an agent that binds TERC-2 by contacting TERC-2 with a test agent and determining whether the agent binds TERC-2. Optionally, the TERC-2 subunit is labeled with a fluorescent label, or a radioactive label. Alternatively, the TERC-2 subunit is attached to a solid phase such as a particle. The particle may be made of metal compounds, silica, latex, polymeric material, or a silica, latex or polymer nuclei coated with a metal or metal compound

The invention also includes an TERC-2 modulator compounds identified according to this screening method, and a pharmaceutical composition which includes the modulators.

Therapeutic Administration

The invention includes administering to a subject a composition comprising a compound that increases or decreases TERC-2 expression or activity (or “therapeutic compound”).

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-inflammatory agents or therapeutic agents for treating, preventing or alleviating a symptom of a particular cell proliferative disorder. A therapeutic regimen is carried out by identifying a mammal, e.g., a human patient suffering from (or at risk of developing) a cell proliferative disorder, using standard methods.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The compound is administered prophylactically, or after the detection of a cell proliferative disorder. The compound is optionally formulated as a component of a cocktail of therapeutic drugs to treat cell proliferative disorders. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the therapeutic compound is formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, suppository, paste, spray, patch, cream, gel, resorbable sponge, or foam. Such formulations are produced using methods well known in the art.

Additionally, compounds are administered by implanting (either directly into a tumor or subcutaneously) a solid or resorbable matrix which slowly releases the compound into adjacent and surrounding tissues of the subject.

For treatment of cardiovascular diseases, the compound is delivered for example to the cardiac tissue (i.e., myocardium, pericardium, or endocardium) by direct intracoronary injection through the chest wall or using standard percutaneous catheter based methods under fluoroscopic guidance for direct injection into tissue such as the myocardium or infusion of an inhibitor from a stent or catheter which is inserted into a bodily lumen. Any variety of coronary catheter, or a perfusion catheter, is used to administer the compound. Alternatively, the compound is coated or impregnated on a stent that is placed in a coronary vessel.

The invention will be further illustrated in the following non-limiting examples.

### EXAMPLE 1

### General Methods

Cell Culture and Stable Expression of shRNA.

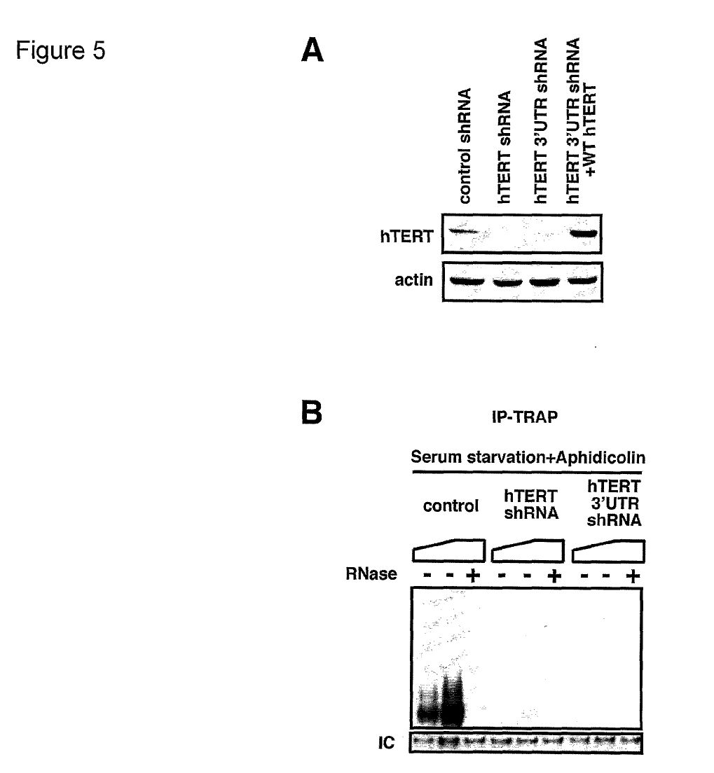

Human diploid fibroblasts were cultured and amphotropic retroviruses were created using replication-defective retroviral vectors as described². To express hTERT-specific shRNA stably in human cells, hTERT sequences from the hTERT coding region (nucleotides 3114 to 3134)² and the hTERT 3′ UTR region (nucleotides 3877 to 3897) into the pMKO.1-puro vector² by introducing oligonucleotides representing hTERT-derived sequences followed by 9 bp to form a loop and the corresponding antisense hTERT nucleotides followed by 5 uridines. The sequences used for the hTERT 3′UTR region hairpin were: 5′-ATTTGGAGTGACCAAAGGTttcaagagaACCTTTGGTCACTCCAAATtttttg-3′ and 5′ aattcaaaaATTTGGAGTGACCAAAGGTtctcttgaaACCTTTGGTCACTCCAAAT-3′, where the capitalized letters represent hTERT sequences. Suppression of hTERT expression by these shRNA is shown in FIG. 5. The control retroviral vector encoding a GFP-specific shRNA was created in pMKO.1-puro with the oligonucleotides 5′-CGCAAGCTGACCCTGAGTTCATTCAAGAGATGAACTTCAGGGTCAGCTTGCTTTTTG 3′ and 5′-AATTCAAAAAGCAAGCTGACCCTGAAGTTCATCTCTTGAATGAACTTCAGGGTCAGC TTGCGGGCC-3′.

### Immunoblotting, Immunofluorescence and Fluorescence In Situ Hybridization (FISH) and RT-PCR.

For indirect immunofluorescence, cells were fixed in chilled acetone, incubated with the indicated primary antibody, washed and then incubated with either AlexaFluor568conjugated or AlexaFluor488-conjugated secondary antibody (Pierce) in 1% BSA for 1 h at 37° C. For telomere-specific FISH, we used a peptide nucleic acid (PNA) probe (CCCTAA)₃ specific for the mammalian telomere sequence (Applied Biosystems). Cells fixed by acetone were hybridized with this telomere-specific PNA probe at 72° C. for 8 min. To remove non-hybridized PNA probes, slides were washed with 0.05% Tween 20 containing PBS at 56° C. for 15 min. Slides were visualized using a Nikon Eclipse E800 fluorescence microscope. No staining was detected when parallel cultures were incuated with a single mismatch (CCCTTA)₃ PNA probe. The antibodies used in this study included: rabbit anti-H2AX (Novus); rabbit anti—H2AX (Upstate Biotechnology); rabbit anti-H2B (Upstate Biotechnology); mouse anti-H3 (Upstate Biotechnology); rabbit anti-H4 (Upstate Biotechnology); rabbit anti-macro H2A.1 (Upstate Biotechnology); rabbit anti-dimethyl H3 (K9) (Upstate Biotechnology); rabbit anti-acetyl H3 (Lys9) (Upstate Biotechnology); rabbit anti-acetyl H4 (K12) (Upstate Biotechnology); goat anti-phospho-specific BRCA1 (Ser1497) (Santa Cruz); rabbit anti-ATM-pS1981 (Rockland); rabbit anti-ATM-S1981 (Rockland); and mouse anti-p53 (Ab6) (Oncogene). Cells were lysed in RIPA buffer (12.5 mM NaPO₄, pH7.2, 2 mM EDTA, 50 mM NaF, 1.25% NP-40, 1.25% SDS, 0.1 mM DTT) except when specific conditions are noted. For extraction under low salt conditions, cells were lysed in a buffer comprised of 20 mM Tris-HCl, pH 7.4, 150 mM NaCl, 0.1% NP40, 0.1 mM DTT. For high salt condition extraction, cells were lysed in a buffer composed of 20 mM Tris-HCl, pH 7.4, 500 mM NaCl, 0.5% NP40, 0.1 mM DTT. For acid precipitation of histones, cells were homogenized in 0.2 N H₂SO₄ and centrifuged. Histones were precipitated by adding ¼ volume of 100% (w/v) TCA. The pellets were suspended in 100% ethanol and centrifuged again at 13,000×g. 10 μg of protein was subjected to immunoblotting. The sequences used for the H2AXR T-PCR were: 5′ TCGGGCCGCGGCAAGACTGGCGGCAA-3′ and 5′-GTACTCCTGGGAGGCCTGGGTGGCCTT-3′. RT was performed on 500 ng of total RNA for 30 min at 42° C. followed by PCR (25 cycles: 94° C. 45 s, 60° C. 45 s, 72° C. 90 s). T-OLA. The telomeric 3′ single-stranded overhang was analyzed by a telomere 3′ overhang assay (T-OLA) as described^{2,12}.

### Micrococcal Nuclease Assay

1×10⁶ cells were suspended in 1 ml nuclei buffer [25 mM HEPES, pH 7.8, 1.5 mM MgCl2, 10 mM KCl, 0.1% NP-40, 1 mM DTT, and protease inhibitor cocktail (Roche)]. Nuclei were obtained by Dounce homogenization (20 strokes, pestle A) and sedimented by centrifugation at 1400×g at 4° C. for 20 min through 1 ml of a solution containing 10 mM Tris-HCl pH 7.4, 15 mM NaCl, 60 mM KCl, 0.15 mM spermine, 0.5 mM spermidine and 10% sucrose. The nuclear pellet was then resuspended in 350 μl of digestion buffer (50 mM Tris-HCl pH 7.5, 15 mM NaCl, 5 mM KCl, 3 mM MgCl₂, 1 mM CaCl₂, 10 mM NaHSO₄, 0.25 M sucrose, 0.15 mM spermine, 0.5 mM spermidine, and 0.15 mM mercaptoethanol) containing micrococcal nuclease (9 U ml⁻¹: Roche). 50 μl from this reaction mixture was mixed with 50 μl of stop solution (200 mM EDTA and 200 mM EGTA pH 7.5) to stop the reaction at the indicated time. Digested DNA was recovered by QIAquick columns (Qiagen), subjected to agarose gel electrophoresis, and visualized by staining with ethidium bromide.

### Clonogenic Assay

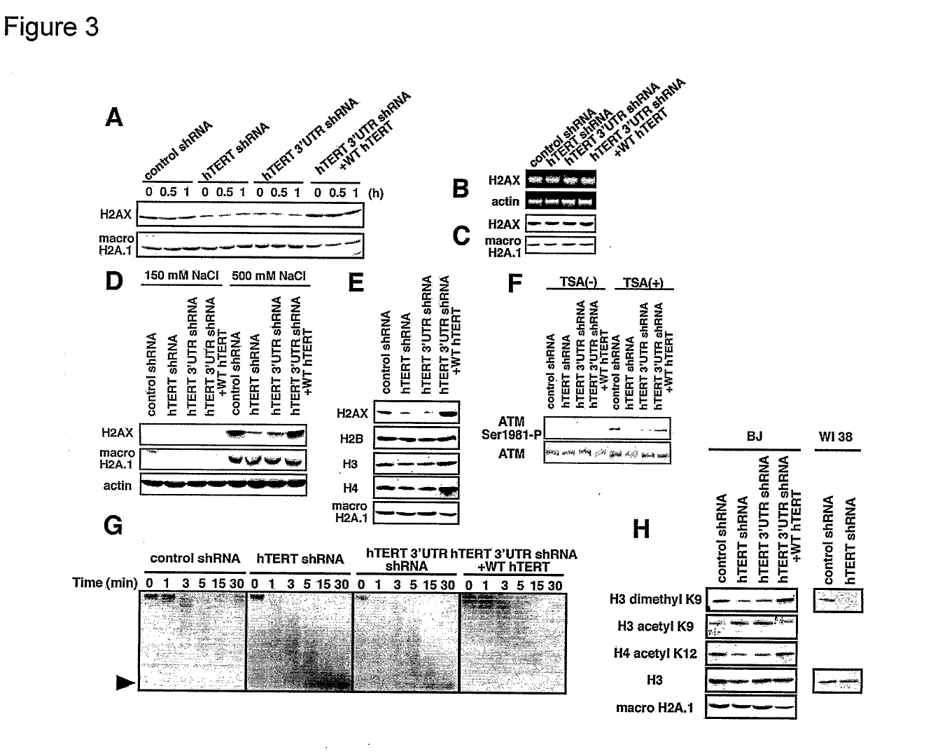

Clonogenic assays were performed using two different seeding protocols. In some experiments, 200 cells were seeded into 9.6 cm² plates in triplicate, and exposed to ionizing radiation after 24-48 h. Cells were allowed to proliferate for 10-12 d, trypsinized and replated into plates to eliminate cell debris. Cells were counted after an additional 5-7 d using a Coulter particle counter (Beckman). In other experiments, 1000 cells were seeded into 9.6 cm² plates in triplicate, irradiated after 24-48 h, incubated 21 days, and stained with crystal violet (0.2%) to identify colonies. Colonies containing greater than 20 cells were counted manually. Identical results were obtained using these two methods, and the experiment shown in FIG. 3F was performed using the first method.

### DNA Repair Assay.

The DNA repair assay was performed as previously described²⁹. Briefly, cells were mock irradiated or irradiated (2 Gy), allowed to recover at 37° C. for 0, 2, and 4 h, trypsinized, and cast into 0.75% Sea-Plaque agarose (FMC). These agarose-cell plugs were placed in lysis buffer (2% sarcosyl, 400 mM EDTA, 1 mg ml⁻¹ proteinase K) and incubated at 50° C. for 38 h, washed with TE buffer, and equilibrated. The plugs were then subjected to pulse field gel electrophoresis using a Biorad Chef 3 apparatus in 0.7% agarose gels, dried, and stained with SYBR Green (Molecular Probes) and the fluorescence signal measured by Image Quant Software as previously described²⁹. The fraction of DNA entering the gel was determined by (signal in lane)/(signal in lane+signal in plug)×100. The relative fraction of DNA breaks repaired at 4 h was determined by calculating the ratio of DNA entering the gel at 4 h to that present immediately after irradiation (0 h). The measured value of signal present in unirradiated cells was subtracted for each sample. The data were normalized to the control shRNA sample and presented as bars representing the mean±standard deviation. Cytogenetic analysis. After exposure to 5 Gy of -radiation, cells were incubated at 37° C. for 24 hrs and subjected to standard cytogenetic protocol as described³⁰. Metaphases were visualized on a Nikon Eclipse E800 microscope, and cytogenetic abnormalities were scored by a blinded observer.

### Analysis of Telomere Structure

Telomere length was measured by hybridizing a ³²P-labeled telomeric (CCCTAA)₃ probe to HinfI- and RsaI-digested genomic DNA. Quantitative-FISH (Q-FISH) analysis was performed as previously described (Martens et al. 1998). Results of Q-FISH analysis are expressed in kilobases as determined by comparison with plasmid DNA containing telomere inserts. The telomeric 3′ single-stranded overhang was analyzed by a telomere 3′ overhang assay (T-OLA) as described (Stewart et al. 2003).

### Cytogenetic Analysis

Prior to or after exposure to 5 Gy of γ-radiation, cells were incubated at 37° C. for 24 h and subjected to a standard cytogenetic protocol (Barch et al. 1997). Cytogenetic abnormalities were scored by a blinded observer using a Nikon Eclipse E800 microscope.

### EXAMPLE 2

### hTERT is a Critical Regulator of the DNA Damage Response Pathway

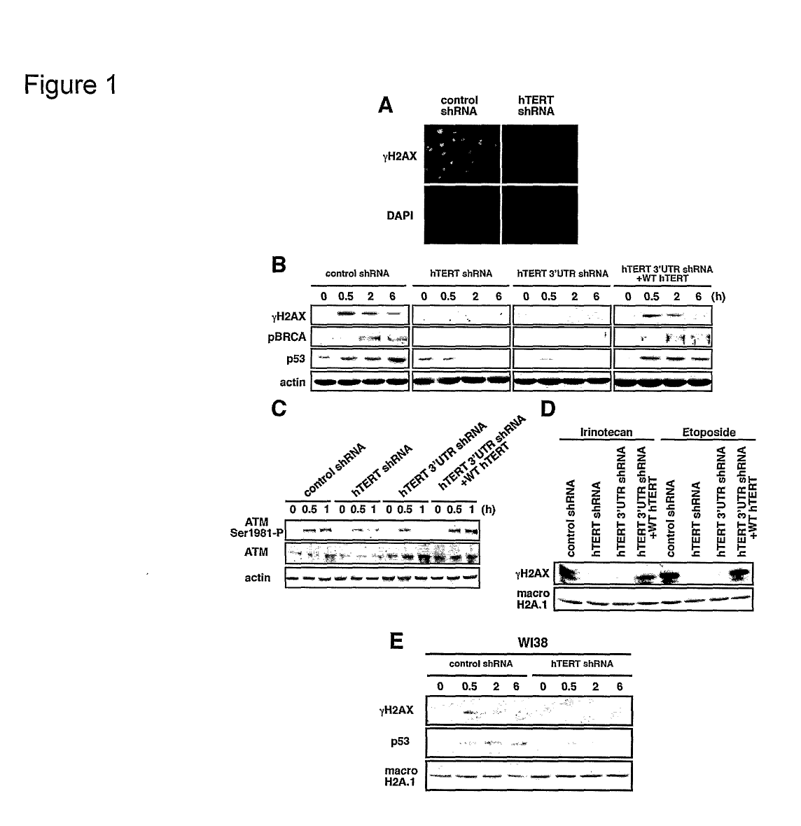

To determine whether telomerase participates in the response to DNA damage, the effect of suppressing hTERT expression on the response to ionizing radiation in diploid human fibroblasts was examined. As expected, irradiation of human BJ fibroblasts expressing a control, green fluorescent protein (GFP)-specific short hairpin (shRNA) vector led to the phosphorylation of H2AX (γ-H2AX) (FIG. 1a,b), phosphorylation of the ATM (FIG. 1c) and BRCA1 tumor suppressor proteins (FIG. 1b), and to the stabilization of the p53 protein (FIG. 1b). Treatment of these fibroblasts with the chemotherapeutic agents irinotecan or etoposide also induced phosphorylation of H2AX (FIG. 1d).

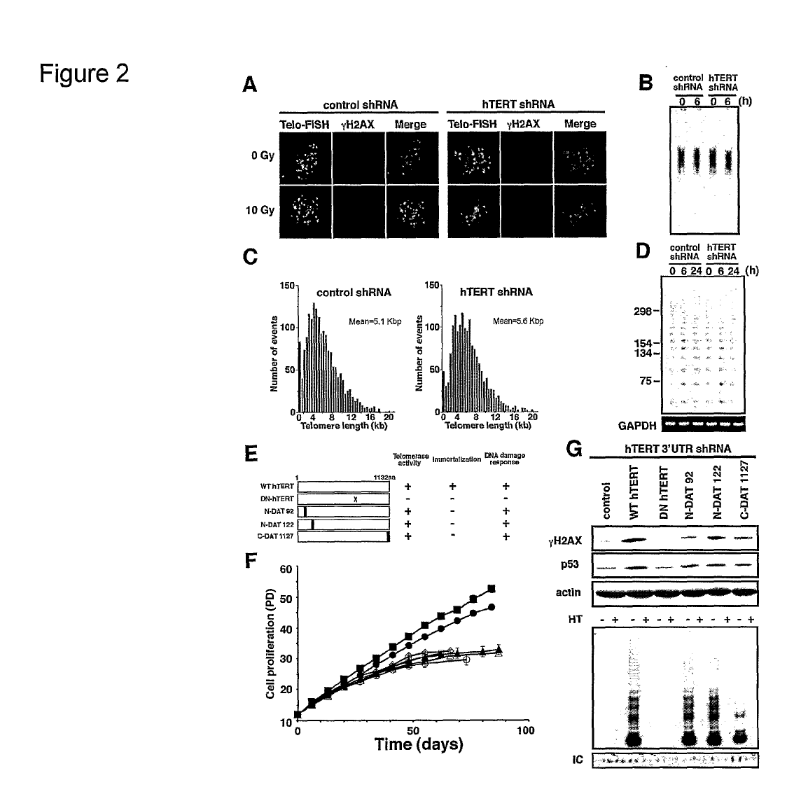

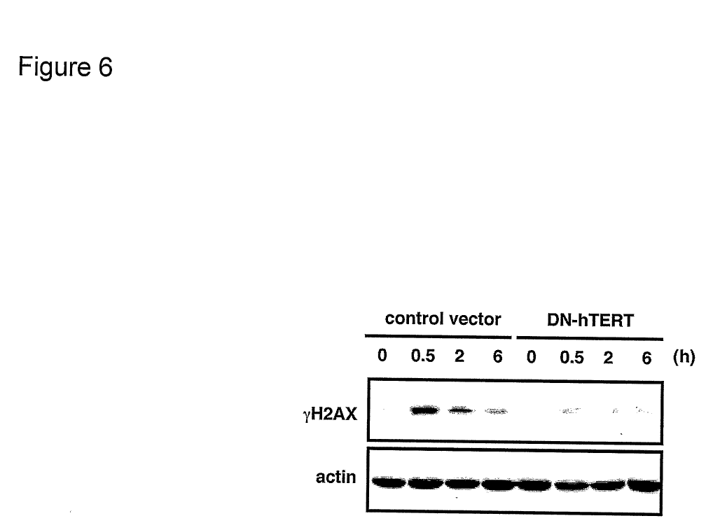

Surprisingly, exposure of parallel cultures of fibroblasts expressing either an hTERT coding sequence-specific shRNA (hTERT shRNA)² or an hTERT 3′untranslated region-specific shRNA (hTERT 3′ UTR shRNA) (FIG. 6) to ionizing radiation, irinotecan or etoposide failed to induce a similar degree of H2AX phosphorylation (FIG. 1a,b,d) or accumulation of NBS-1 in nuclear foci (data not shown). In addition, the autophosphorylation of ATM was diminished (FIG. 1c), and the phosphorylation of BRCA1 or the stabilization of p53 protein levels in cells lacking hTERT expression (FIG. 1b) was not observed. These findings indicate that the DNA damage response in cells lacking hTERT is impaired. Expression of wildtype hTERT (WT hTERT) in cells expressing the hTERT 3′ UTR-specific shRNA rescued telomerase activity (FIG. 2e) and permitted cells to respond to DNA damage (FIG. 1b,c,d). Treatment of fibroblasts expressing a catalytically inactive hTERT mutant (DN hTERT), which inhibits the catalytic activity of telomerase¹¹, to ionizing radiation also impaired the DNA damage response (FIG. 7). Thus, loss of hTERT function abrogates the cellular response to DNA damage, implicating hTERT as a critical regulator of the DNA damage response pathway. Although overexpression of hTERT stabilizes telomere length in human cells¹, alterations in overall telomere length (FIG. 2a and data not shown) or changes in the length of the 3′ telomeric single-stranded overhang¹² were not detected after irradiation of cells expressing an hTERT specific shRNA as compared to cells expressing a control shRNA over the short time periods encompassed by these experiments (FIG. 2b). Moreover, less than 10% of telomeres co-localized with nuclear foci containing —H2AX after treatment with ionizing radiation (FIG. 2a). In addition, although suppression of hTERT expression induces premature entry into senescence in human fibroblasts², these studies were performed in parallel, exponentially dividing cultures at early passage (population doubling 12) to ensure that the DNA damage response observed in senescent cells did not contribute to these experiments. Indeed, in unirradiated cells, we failed to identify evidence of karyotypic abnormalities in cells expressing either the control shRNA or an hTERT-specific shRNA prior to irradiation (See legend to Table 1), confirming that the suppression of hTERT in early passage fibroblasts does not, by itself, result in immediate telomere dysfunction.

### EXAMPLE 3

### hTERT Modulation of the DNA Damage Response is Independent of Telomere Elongation

To determine whether the telomere elongation function of hTERT was required for the DNA damage response, several hTERT mutants into cells were introduced in which the endogenous hTERT was suppressed by the expression of the hTERT 3′ UTR-specific shRNA. Specifically, hTERT mutants were expressed that harbor mutations in the amino-(N) and carboxy (C)-terminal DAT (dissociates activities of telomerase) domains (N-DAT92, N-DAT122, C-DAT1127) as well as the DN hTERT mutant (FIG. 2g)^{11,14-16}. These DAT mutants have previously been shown to reconstitute telomerase biochemical activity yet fail to elongate telomeres or to confer an immortal phenotype when expressed in human cells¹⁴⁻¹⁶. These hTERT mutants exhibited telomerase activity (FIG. 2g) and failed to rescue the premature senescence phenotype found in human fibroblasts that lack endogenous hTERT expression (FIG. 2f)². Despite this defect in telomere maintenance, these hTERT mutants restored the ability of human fibroblasts to phosphorylate H2AX and stabilize p53 after exposure to ionizing radiation (FIG. 2g). note that N-DAT92 only partially rescues the DNA damage response (FIG. 2g); this hTERT mutant also exhibits catalytic defects when assessed in telomerase assays that are not based on PCR amplification¹⁶. Hence, hTERT does not appear to act primarily by elongating overall telomere length to modulate the DNA damage response.

### EXAMPLE 4

### Suppression of hTERT Expression Modulates Overall Chromatin Architecture

Phosphorylation of H2AX plays an important role in the response to DNA damage and is involved in both homologous recombination and non-homologous end joining^{17,18}. Since suppression of hTERT expression led to a profound defect in H2AX phosphorylation H2AX levels in fibroblasts expressing control or either of the two hTERT-specific shRNAs was examined. When cells were lysed in detergent-based buffers over a wide range of salt concentrations, we detected 75% less H2AX protein in whole cell lysates derived from cells lacking hTERT (FIG. 3a,d). This decrease in soluble H2AX was not the result of altered H2AX transcription (FIG. 3b) but instead correlated with enhanced association of H2AX with the insoluble cell fraction. When whole cell proteins were precipitated under acidic conditions, equal amounts of H2AX in cells that expressed or lacked hTERT (FIG. 3c) were recovered. In contrast, differences in the amounts of soluble macro H2A.1 H₂B, H3, and H4 in cells that expressed or lacked hTERT expression (FIG. 3d,e) were not detected. Although increased levels of H3 and H4 in cells overexpressing hTERT were consistently found (FIG. 3e).

Autophosphorylation of ATM occurs rapidly in response to changes in chromatin structure induced by exposure to agents such as trichostatin A (TSA), even in the absence of DNA double strand breaks¹⁹. To determine if hTERT suppression also affected the activation of ATM after treatment with TSA, cells were treated that express or lack hTERT and found that ATM phosphorylation induced by TSA treatment was also significantly impaired (FIG. 3f). These findings suggest that suppression of hTERT expression modulates overall chromatin architecture. To investigate this possibility further, nuclear preparations from cells expressing or lacking hTERT with micrococcal nuclease were treated and it was found that chromatin derived from cells lacking hTERT was significantly more sensitive to micrococcal nuclease treatment compared to control cell lines (FIG. 3g).

### EXAMPLE 4

### Suppression of hTERT Expression Effects Post Translational Modification of Histone Tails

Consistent with the finding that loss of hTERT expression affected overall sensitivity of chromatin to micrococcal nuclease treatment, it was found that particular post-translational modifications of histone tails were also affected by hTERT suppression. Specifically, it was found that decreased levels of histone H3-lysine (K) 9 dimethylation and increased amounts of H3-K9 acetylation in cells lacking hTERT (FIG. 3h). The heterochromatic proteins 1 (HP1) associate with di- and tri-methylated but not acetylated forms of H3-K9 to form heterochromatin²¹. In assessing other histone modifications that may be important for heterochromatin organization, it was shown that the degree of H4-K12 acetylation was also decreased in cells lacking hTERT expression (FIG. 3hc). The combination of decreased H3-K9 dimethylation and H4-K12 acetylation is reminiscent of that seen in Suv39h histone methyltransferase deficient cells, which also exhibit impaired genomic stability²². These observations suggest that loss of hTERT expression alters the overall state of chromatin into a configuration that inhibits the activation of the DNA damage response. These observations suggest that suppression of hTERT expression alters H2AX solubility.

### EXAMPLE 5

### hTERT Plays a Role in Cellular Repair to Genotoxic Damage Histone Tails

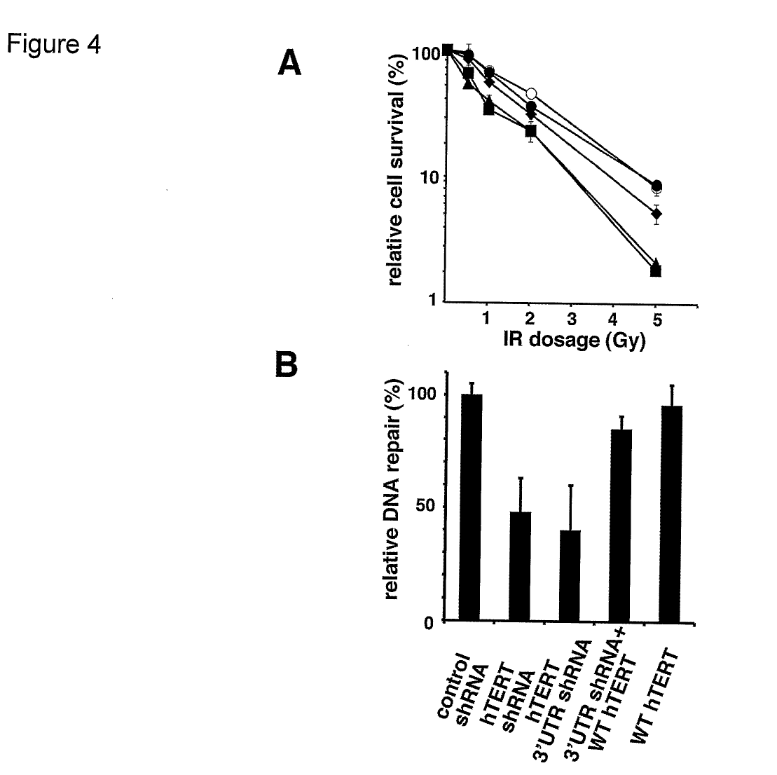

Since loss of even one copy of H2AX^{17,18} dramatically impairs the DNA damage response and affects genome stability, we ascertained the functional consequences of treating human fibroblasts unable to express hTERT with ionizing radiation. Cells expressing either of the two hTERT-specific shRNAs showed a significant increase in their sensitivity to ionizing radiation, as assessed in clonogenic growth assays (FIG. 4a). Co-expression of WT hTERT in cells expressing the hTERT 3′ UTR-specific shRNA rescued this increased sensitivity to ionizing irradiation (FIG. 4a). In consonance with these findings, it was found that suppression of hTERT expression also altered the capacity of these cells to repair DNA after treatment with ionizing radiation, as assessed by the electrophoretic migration rates of genomic DNA into pulse-field agarose gels (FIG. 4b). Finally, human cells lacking hTERT expression rapidly accumulated statistically significant increased numbers of chromosomal fragments compared to cells expressing hTERT either transiently (vector control) or constitutively (WT hTERT) (Table 1). These findings demonstrate that hTERT plays a functionally important role in allowing cells to repair genotoxic damage.

TABLE 1
Statistical analysis of cytogenetic abnormalities.

| Comparison group | Comparison values | P value |
|---|---|---|
| Number of fragments per metaphase | | |
| WT hTERT vs. Vector control | 0.524 vs. 0.718 | 0.41 |
| Vector control vs. hTERT shRNA | 0.718 vs. 1.31 | 0.02 |
| WT hTERT vs. hTERT shRNA | 0.524 vs. 1.31 | 0.008 |
| Proportion of normal metaphases | | |
| WT hTERT vs. Vector control | 0.619 vs. 0.462 | 0.25 |
| Vector control vs. hTERT shRNA | 0.462 vs. 0.241 | 0.058 |
| WT hTERT vs. hTERT shRNA | 0.619 vs. 0.241 | 0.008 |

### EXAMPLE 6

### Identification of RNAs Associated with Telomerase

A sequence encoding for a Flag and HA tag were fused to the N-terminus of the hTERT cDNA in a pBABE retroviral construct containing a blasticidin resistance marker (FH hTERT). Retroviruses containing this FH HTERT construct were generated and used to infect wild type human BJ fibroblasts. These fibroblasts were selected for those that stably expressed the retroviral construct by blasticidin selection. Expression of the full length FH HTERT protein was confirmed by immunoprecipitation with an anti-flag antibody (Flag-M2 Sigma) and immunoblotting with an HA-11 antibody (covance). Functionality of FH HTERT was confirmed by immunoprecipitation with the anti-flag antibody and subjecting the immunoprecipitated protein to a PCR based telomerase repeat amplification protocol.

RNA identification was accomplished by lysing one 15 cm plate of near confluent cells in 700 uL lysis buffer (20 mM Tris pH 7.4, 150 mM NaCl, 0.5% NP-40, one Roche mini-complete protease inhibitor tablet per 10 mL, 3 uL Invitrogen RNase OUT per 10 mL), homogenizing by passing through a pipette tip and incubating for 30 minutes on ice. Lysates were centrifuged at 4 C and 13,000×g for 10 minutes and the supernatant was removed to a new tube. To the cleared lysate was added 25 uL of Flag M2 agarose beads (50% solution equilibrated with lysis buffer, Sigma), 25 uL of HA-11 crosslinked Protein G Sepharose Beads, or 25 uL of Protein G Sepharose beads (50% solution equilibrated with lysis buffer, Sigma) plus 1 ug of an anti-actinin antibody. The beads were allowed to incubate with the lysate for 1 hour at 4 C with rotation. After one hour, the beads were pelleted at 4 C and 1000×g for 1 minute and the supernatant was discarded and replaced with 1 mL lysis buffer. This was repeated three times with gentle mixing between each addition of lysis buffer followed by three times with 5 minutes of rotation at 4 C between each addition of lysis buffer for a total of 6 washes. After the final was, the beads were pelleted and supernatant discarded and the beads were subjected to RNA purification by the RNeasy Mini Kit (Qiagen), treating the beads as a 30 uL reaction with a final elution volume of 30 uL. The resulting RNA was ligated to a phosphorylated primer of the sequence ACTCTGCGTTGATACCACTGCTT with a 3′ inverted thymidine. Specifically, to the 30 uL of RNA was added 3.5 uL 10 T4 RNA ligase buffer (NEB), 1 uL T4 RNA ligase (NEB), 0.5 uL RNase OUT, 2 uL 100 uM primer. This ligation was carried out at 37 C for one hour. Following the ligation, the reaction was subjected to RNA purification by the RNeasy Mini Kit with a final elution volume of 30 uL. The ligated RNA was then subjected to RT-PCR using the SuperScript One-Step RT-PCR kit (Invitrogen) using a primer complementary to the ligated primer (AAGCAGTGGTATCAACGCAGAGT, Primer IIA, BD Biosciences Clontech). Specifically, to the 30 uL of RNA was added 2 uL 20 uM Primer IIA and this was heated to 70 C. for 3 minutes followed by cooling to 4 C for 2 minutes. To this was added 35 uL 2× reaction buffer, 0.5 uL RNase OUT, 1 uL RT/Taq mix, and 2 uL 20 uM of a second primer (AAGCAGTGGTATCAACGCAGAGTGGG). This reaction was incubated for 25 minutes at 42 C, 2 minutes at 94 C and then 40 cycles of PCR (94 C 15″, 55 C 30″, 72 C 1′).

The resulting product was analyzed on a 1.2.% agarose/TAE gel. Bands in common between the HA and FLAG precipitated lanes, but not occurring in the actinin precipitated lane were isolated for further study.

### REFERENCES

1. Bodnar, A. G. et al. Extension of life-span by introduction of telomerase into normal human cells. Science 279, 349-52 (1998).
2. Masutomi, K. et al. Telomerase maintains telomere structure in normal human cells. Cell 114, 241-53 (2003).
3. Gonzalez-Suarez, E. et al. Increased epidermal tumors and increased skin wound healing in transgenic mice overexpressing the catalytic subunit of telomerase, mTERT, in basal keratinocytes. Embo J 20, 2619-30. (2001).
4. Artandi, S. E. et al. Constitutive telomerase expression promotes mammary carcinomas in aging mice. Proc Natl Acad Sci USA 99, 8191-6 (2002).
5. Stewart, S. A. et al. Telomerase contributes to tumorigenesis by a telomere length-independent mechanism. Proc Natl Acad Sci USA 99, 12606-12611 (2002).
6. Wright, W. E. & Shay, J. W. Cellular senescence as a tumor-protection mechanism: the essential role of counting. Curr Opin Genet Dev 11, 98-103. (2001).
7. Serrano, M., Lin, A. W., McCurrach, M. E., Beach, D. & Lowe, S. W. Oncogenic ras provokes premature cell senescence associated with accumulation of p53 and p16 NK4a. Cell 88, 593-602. (1997).
8. von Zglinicki, T., Pilger, R. & Sitte, N. Accumulation of single-strand breaks is the major cause of telomere shortening in human fibroblasts. Free Radic Biol Med 28, 64-74 (2000).
9. Parrinello, S. et al. Oxygen sensitivity severely limits the replicative lifespan of murine fibroblasts. Nat Cell Biol 5, 741-7 (2003).
10. Schmitt, C. A. et al. A senescence program controlled by p53 and p16INK4a contributes to the outcome of cancer therapy. Cell 109, 335-46 (2002).
11. d'Adda di Fagagna, F. et al. A DNA damage checkpoint response in telomere-initiated senescence. Nature 426, 194-8 (2003).
12. Takai, H., Smogorzewska, A. & de Lange, T. DNA damage foci at dysfunctional telomeres. Curr Biol 13, 1549-56 (2003).
13. Bakkenist, C. J., Drissi, R., Wu, J., Kastan, M. B. & Dome, J. S. Disappearance of the telomere dysfunction-induced stress response in fully senescent cells. Cancer Res 64, 3748-52 (2004).
14. Chan, S. W. & Blackburn, E. H. New ways not to make ends meet: telomerase, DNA damage proteins and heterochromatin. Oncogene 21, 553-63 (2002).
15. Forsythe, H. L., Elmore, L. W., Jensen, K. O., Landon, M. R. & Holt, S. E. Retroviral-mediated expression of telomerase in normal human cells provides a selective growth advantage. Int J Oncol 20, 1137-43 (2002).
16. Kang, H. J. et al. Ectopic expression of the catalytic subunit of telomerase protects against brain injury resulting from ischemia and NMDA-induced neurotoxicity. J Neurosci 24, 1280-7 (2004).
17. Hahn, W. C. et al. Inhibition of telomerase limits the growth of human cancer cells. Nat Med 5, 1164-70. (1999).
18. Stewart, S. A. et al. Erosion of the telomeric single-strand overhang at replicative senescence. Nat Genet. 33, 492-6 (2003).
19. Sedelnikova, O. A. et al. Senescing human cells and ageing mice accumulate DNA lesions with unrepairable double-strand breaks. Nat Cell Biol 6, 168-70 (2004).
20. Armbruster, B. N., Banik, S. S., Guo, C., Smith, A. C. & Counter, C. M. N-terminal domains of the human telomerase catalytic subunit required for enzyme activity in vivo. Mol Cell Biol 21, 7775-86 (2001).
21. Banik, S. S. et al. C-terminal regions of the human telomerase catalytic subunit essential for in vivo enzyme activity. Mol Cell Biol 22, 6234-46 (2002).
22. Lee, S. R., Wong, J. M. & Collins, K. Human telomerase reverse transcriptase motifs required for elongation of a telomeric substrate. J Biol Chem 278, 52531-6 (2003).
23. Celeste, A. et al. Genomic instability in mice lacking histone H2AX. Science 296, 922-7 (2002).
24. Bassing, C. H. et al. Increased ionizing radiation sensitivity and genomic instability in the absence of histone H2AX. Proc Natl Acad Sci USA 99, 8173-8 (2002).
25. Bakkenist, C. J. & Kastan, M. B. DNA damage activates ATM through intermolecular autophosphorylation and dimer dissociation. Nature 421, 499-506 (2003).
26. Tommerup, H., Dousmanis, A. & de Lange, T. Unusual chromatin in human telomeres. Mol Cell Biol 14, 5777-85 (1994).
27. Nakayama, J., Rice, J. C., Strahl, B. D., Allis, C. D. & Grewal, S. I. Role of histone H3 lysine 9 methylation in epigenetic control of heterochromatin assembly. Science 292, 110-3 (2001).
28. Lachner, M., O'Carroll, D., Rea, S., Mechtler, K. & Jenuwein, T. Methylation of histone H3 lysine 9 creates a binding site for HP1 proteins. Nature 410, 116-20 (2001).
29. Bannister, A. J. et al. Selective recognition of methylated lysine 9 on histone H3 by the HP1 chromo domain. Nature 410, 120-4 (2001).
30. Celeste, A. et al. H2AX haploinsufficiency modifies genomic stability and tumor susceptibility. Cell 114, 371-83 (2003).
31. Bassing, C. H. et al. Histone H2AX: a dosage-dependent suppressor of oncogenic translocations and tumors. Cell 114, 359-70 (2003).
32. Peters, A. H. et al. Loss of the Suv39h histone methyltransferases impairs mammalian heterochromatin and genome stability. Cell 107, 323-37 (2001).
33. Kramer, K. M. & Haber, J. E. New telomeres in yeast are initiated with a highly selected subset of TG1-3 repeats. Genes Dev 7, 2345-56 (1993).
34. Myung, K., Datta, A. & Kolodner, R. D. Suppression of spontaneous chromosomal rearrangements by S phase checkpoint functions in Saccharomyces cerevisiae. Cell 104, 397-408 (2001).
35. Stellwagen, A. E., Haimberger, Z. W., Veatch, J. R. & Gottschling, D. E. Ku interacts with telomerase RNA to promote telomere addition at native and broken chromosome ends. Genes Dev 17, 2384-95 (2003).
36. Flint, J. et al. Healing of broken human chromosomes by the addition of telomeric repeats. Am J Hum Genet. 55, 505-12 (1994).
37. Sprung, C. N., Reynolds, G. E., Jasin, M. & Murnane, J. P. Chromosome healing in mouse embryonic stem cells. Proc Natl Acad Sci USA 96, 6781-6 (1999).
38. Rouse, J. & Jackson, S. P. Interfaces between the detection, signaling, and repair of DNA damage. Science 297, 547-51 (2002).
39. Narita, M. et al. Rb-mediated heterochromatin formation and silencing of E2F target genes during cellular senescence. Cell 113, 703-16 (2003).
40. Shin, K. H. et al. Introduction of human telomerase reverse transcriptase to normal human fibroblasts enhances DNA repair capacity. Clin Cancer Res 10, 2551-60 (2004).
41. Wong, K. K. et al. Telomere dysfunction impairs DNA repair and enhances sensitivity to ionizing radiation. Nat Genet. 26, 85-8. (2000).
42. Barch, M. J., Knutsen, T. & Spurbeck, J. L. (eds.) The AGT Cytogenetics Laboratory Manual (Lippincott-Raven, Philadelphia, N.Y., 1997).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A telomerase RNA subunit comprising at least 100 nucleotides of SEQ ID NO:1, wherein said subunit binds a telomerase catalytic subunit (TERT) polypeptide.

2. The RNA subunit of claim 1, wherein said subunit is at least 250 nucleotides in length.

3. The RNA subunit of claim 1, wherein said subunit is at least 500 nucleotides in length.

4. The RNA subunit of claim 1, wherein said subunit is at least 1000 nucleotides in length.

5. The RNA subunit of claim 1, wherein said catalytic subunit polypeptide is human or murine.

6. A vector comprising the nucleic acid of claim 1.

7. A cell comprising the vector of claim 6.

8. A complex comprising a telomerase catalytic subunit (TERT) polypeptide and the RNA subunit of claim 1.

9. The complex of claim 8, wherein said catalytic subunit polypeptide is human or murine.

10. A method of inducing cell senescence comprising contacting a cell with a compound that inhibits the interaction between a telomerase catalytic subunit (TERT) polypeptide and the RNA subunit of claim 1.

11. The method of claim 10, further comprising contacting the cell with a cytotoxic agent.

12. The method of claim 11, wherein said cytotoxic agent is a chemotherapeutic compound.

13. The method of claim 10, wherein said cell is contacted in vivo, in vitro or ex vivo.

14. The method of claim 10, wherein said cell is a cancer cell.

15. The method of claim 10, where said compound is an anti-TERC2 antibody.

16. A method of inducing cell senescence comprising contacting a cell with a compound that decreases the activity or expression of TERC-2.

17. The method of claim 16, wherein said compound is a TERC-2 anti-sense nucleic acid or a TERC-2 RNAi.

18. A method of enhancing cell viability comprising contacting a cell with a composition comprising the RNA subunit of claim 1.

19. A method identifying an inhibitor of the telomerase-TERC-2 subunit interaction comprising:
a) bringing into contact a telomerase protein, a TERC-2 RNA and a test compound under conditions where the telomerase protein and the TERC-2 RNA, in the absence of compound, are capable of forming a complex; and
b) determining the amount of complex formation
wherein a decrease in the amount of complex formation in the presence of the test compound compared to the absence of the test compound indicates said compound is an inhibitor of the telomerase-TERC-2 subunit interaction.

20. A method identifying an enhancer of the telomerase-TERC-2 subunit interaction comprising:
a) bringing into contact a telomerase protein, a TERC-2 RNA and a test compound under conditions where the telomerase protein and the TERC-2 RNA, in the absence of compound, are capable of forming a complex; and
b) determining the amount of complex formation
wherein an increase in the amount of complex formation in the presence of the test compound compared to the absence of the test compound indicates said compound is an enhancer of the telomerase-TERC-2. subunit interaction.

21. A method of identifying an agent that binds to telomerase RNA subunit of claim 1, the method comprising:
(a) introducing said subunit to said agent; and
(a) determining whether said agent binds to said subunit.

22. The method of claim 21, wherein said subunit comprises a label.

23. The method of claim 22, wherein said label is a fluorescent label, or a radioactive label.
